(19) United States
(12) Patent Application Publication
(10) Pub. No.: US 2009/0215787 A1
(43) Pub. Date: Aug. 27, 2009

(54) Preparations of new polymorphic forms of varenicline tartrate

(75) Inventors: Cai Gu Huang, Guangzhou (CN); Hui Min He Huang, Guangzhou (CN)

Correspondence Address: Mai De Ltd., P.O. Box 193, Northborough, MA, 01532, US

(73) Assignee: Mai De Ltd.

(21) Appl. No.: 12/321,157

(22) Filed: Jan. 21, 2009

Related U.S. Application Data
(60) Provisional application No. 61/066,494, filed on Feb. 22, 2008.
(60) Provisional application No. 61/128,073, filed on May 19, 2008.

(51) Int. Cl. A61K 31/4995; C07D 495/08; A61P 25/34

(52) U.S. Cl. 514/250; 544/345

(57) ABSTRACT

The present invention is directed to an amorphous form, three novel polymorph form of crystalline varenicline tartrate, namely Form D, Form E and Form F. The present invention also provides processes of their preparations and pharmaceutical composition comprising such material and their use in therapy. Form D is new anhydrous varenicline tartrate, and can be prepared from recrystallizing varenicline tartrate in a mixture of methanol and water or a mixture of N,N-dimethylformamide and water. Form E is a new varenicline tartrate monohydrate, and can be prepared recrystallizing varenicline tartrate in a mixture of isopropanol and water. Form F is another new varenicline tartrate monohydrate, and can be prepared recrystallizing varenicline tartrate in a mixture of acetone and water. The X-ray powder diffraction pattern (X-RPD), Fourier transform infrared (FT-IR), differential scanning calorimetry (DSC) and thermogravimetric analysis (TGA) techniques are used to characterize amorphous form and crystalline polymorphic forms.

This application claims the benefit of priority to U.S. Provisional Patent Application Ser. No. 61/066,494 filed on Feb. 22, 2008 and U.S. Provisional Patent Application Ser. No. 61/128,073 filed on May 19, 2008, the disclosure of which is hereby incorporated by reference.

### FIELD OF THE INVENTION

The present invention is directed to a novel amorphous form and three novel crystalline polymorphic forms of varenicline tartrate (Form D, Form E and Form F), to processes for preparing said polymorphic forms, to pharmaceutical compositions comprising the same, and to methods of treatment using the same.

### BACKGROUND OF THE INVENTION

Varenicline tartrate is the L-tartrate salt of 5,8,14-triazatetracyclo [10.3.1.0^{2,11}.0^{4,9}]-hexadeca-2(11),3,5,7,9-pentaene, and its chemical structure is shown as follows:

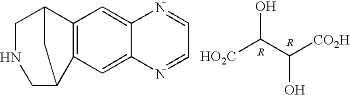

Varenicline and its acid addition salts thereof are disclosed in U.S. Pat. No. 6,410,500 (or WO 99/35131).

Varenicline and its pharmaceutically salts such as varenicline tartrate are useful in the treatment of inflammatory bowel disease (including but not limited to ulcerative colitis, pyoderma gangrenosum and Crohn's disease), irritable bowel syndrome, spastic dystonia, chronic pain, acute pain, celiac sprue, pouchitis, vasoconstriction, anxiety, panic disorder, depression, bipolar disorder, autism, sleep disorders, jet lag, amyotrophic lateral sclerosis (ALS), cognitive dysfunction, hypertension, bulimia, anorexia, obesity, cardiac arrhythmias, gastric acid hypersecretion, ulcers, pheochromocytoma, progressive supranuclear palsy, chemical dependencies and addictions (e.g., dependencies on, or addictions to nicotine (and/or tobacco products), alcohol, benzodiazepines, barbiturates, opioids or cocaine), headache, migraine, stroke, traumatic brain injury (TBI), obsessive-compulsive disorder (OCD), psychosis, Huntington's chorea, tardive dyskinesia, hyperkinesia, dyslexia, schizophrenia, multi-infarct dementia, age-related cognitive decline, epilepsy, including petit mal absence epilepsy, senile dementia of the Alzheimer's type (AD), Parkinson's disease (PD), attention deficit hyperactivity disorder (ADHD) and Tourette's Syndrome, particularly, nicotine dependency, addition and withdrawal; including use in smoking cessation therapy.

Varenicline tartrate salts (including L-tartrate, D, L-tartrate and D-tartrate salts) and their polymorphic forms are disclosed in U.S. Pat. No. 6,890,927 and U.S. Pat. No. 7,265,119. In particular, three polymorphic forms of varenicline L-tartrate, Form A, Form B and Form C were disclosed in these patents. Among them, Form A and Form B are anhydrous varenicline L-tartrate, and Form C is varenicline L-tartrate hydrate.

New crystalline polymorph of a drug substance may display different melting point, hygroscopicity, stability, solubility and/or dissolution rate, crystallinity, crystal habits, bioavailability and formulation handling characteristics, which are among the numerous properties that need to be considered in preparing medicament that can be effectively administered. Therefore, the regulatory agencies require a definitive control of polymorphic form of the active component in solid pharmaceutical dosage forms.

The known polymorphic forms of varenicline L-tartrate (varenicline tartrate or Compound I thereafter) discovered in the prior art are not very stable and may intend to convert into other polymorphic forms under certain storage and manufacturing conditions. The disadvantages of known polymorphic forms of varenicline tartrate render them less useful or favorable for preparing pharmaceutical formulations or bulk handling.

Accordingly, there is an ongoing need to search new polymorphic forms of varenicline tartrate that may have better stability, good material flow character, and lower water or solvent residue contents and thus offer advantages for preparing reproducible pharmaceutical formulations. The novel and new polymorphic forms of varenicline tartrate in the present invention help fulfill this and other needs.

### SUMMARY OF THE INVENTION

The present invention is directed to three novel polymorphic forms of L-tartrate salt of 5,8,14-triazatetracyclo[10.3.1.0^{2,11}.0^{4,9}]-hexadeca-2(11),3,5,7,9-pentaene (varenicline tartrate or Compound I). The novel polymorphic forms of the present invention, namely Form D, Form E and Form F, display distinct physicochemical characteristics, which may have advantages in the preparation of certain pharmaceutical compositions of Compound I, relative to other known crystalline forms of varenicline tartrate.

Thus as a first aspect, the present invention provides a novel polymorphic form (Form D) of L-tartrate salt of 5,8,14-triazatetracyclo[10.3.1.0^{2,11}.0^{4,9}]-hexadeca-2(11),3,5,7,9-pentaene (varenicline tartrate or Compound I).

In another aspect, the present invention provides a composition or drug substance comprising (a) polymorph Form D of varenicline tartrate and (b) a crystalline, hydrate, solvate, amorphous, polymorph Form A, Form B, Form C, Form E, Form F or other polymorphic forms of varenicline tartrate other than Form D, wherein the total weight of varenicline tartrate in the composition is the sum of (a) and (b).

In a yet another aspect, the present invention provides processes for preparing Form D of varenicline tartrate by recrystallizing varenicline tartrate in a mixture of methanol, N,N-dimethyformamide (DMF) and water, followed by isolating and drying the product.

In a still aspect, the present invention provides a novel polymorphic form (Form E) of L-tartrate salt of 5,8,14-triazatetracyclo[10.3.1.0^{2,11}.0^{4,9}]-hexadeca-2(11),3,5,7,9-pentaene (varenicline tartrate or Compound I).

In another aspect, the present invention provides a composition or drug substance comprising (a) polymorph Form E of varenicline tartrate and (b) a crystalline, hydrate, solvate, amorphous, polymorph Form A, Form B, Form C, Form D, Form F or other polymorphic forms of varenicline tartrate other than Form E, wherein the total weight of varenicline tartrate in the composition is the sum of (a) and (b).

In a yet another aspect, the present invention provides processes for preparing Form E of varenicline tartrate by recrystallizing varenicline tartrate in a mixture of isopropanol (IPA) and water, followed by isolating and drying the product.

In a further aspect, the present invention provides a novel polymorphic form (Form F) of L-tartrate salt of 5,8,14-triazatetracyclo[10.3.1.0^{2,11}.0^{4,9}]-hexadeca-2(11),3,5,7,9-pentaene (varenicline tartrate or Compound I).

In another aspect, the present invention provides a composition or drug substance comprising (a) polymorph Form F of varenicline tartrate and (b) a crystalline, hydrate, solvate, amorphous, polymorph Form A, Form B, Form C, Form D, Form E or other polymorphic forms of varenicline tartrate other than Form F, wherein the total weight of varenicline tartrate in the composition is the sum of (a) and (b).

In a yet another aspect, the present invention provides processes for preparing Form F of varenicline tartrate by recrystallizing varenicline tartrate in a mixture of acetone and water, followed by isolating and drying the product.

In another aspect, the present invention accordingly provides a pharmaceutical composition comprising Form D of varenicline tartrate and one or more pharmaceutically acceptable diluents or carriers and, optionally, one or more other physiologically active agents.

In still another aspect, the present invention accordingly provides a pharmaceutical composition comprising Form E of varenicline tartrate and one or more pharmaceutically acceptable diluents or carriers and, optionally, one or more other physiologically active agents.

In a further aspect, the present invention accordingly provides a pharmaceutical composition comprising Form F of varenicline tartrate and one or more pharmaceutically acceptable diluents or carriers and, optionally, one or more other physiologically active agents.

The present invention is further directed to a novel amorphous form of the L-tartrate salt of 5,8,14-triazatetracyclo[10.3.1.0^{2.11}.0^{4,9}]-hexadeca-2(11),3,5,7,9-pentaene (varenicline tartrate or Compound I). The amorphous form of the present invention displays distinct dissolution characteristics relative to crystalline forms of the tartrate salt of 5,8,14-triazatetracyclo [10.3.1.0^{2,11}.0^{4,9}]-hexadeca-2(11),3,5,7,9-pentaene which may have advantages in the preparation of certain pharmaceutical compositions of Compound I. Amorphous forms of Compound I may also exhibit distinct bioavailability and other pharmacokinetic characteristics compared to crystalline forms rendering them preferred forms for certain clinical applications.

The present invention further provides a composition and/or drug substance comprising an amorphous form of varenicline tartrate in amount from 3% to 100% weight by weight.

The present invention also concerns pharmaceutical compositions comprising the amorphous form of varenicline tartrate with one or more pharmaceutically acceptable excipients.

The present invention further provides a process for preparing the amorphous form of varenicline tartrate by using freeze-drying or spray-drying technique.

The present invention still provides a solid amorphous dispersion of varenicline tartrate and a carrier. The said carrier is selected from a group of excipients including solid polyethylene glycol (PEG), polyvinylpyrrolidone (PVP) and its derivatives, lactose, starches, mannitol, methylcellulose, hydroxylmethylcelluse, ethylcellulose, hydroxyethylcellulose, hydroxylpropylcellulose, hydroxylpropylmethylcellulose (HPMC), α-cyclodextrin, β-cyclodextrin or hydroxylpropyl-β-cyclodextrin and their derivatives.

The present invention still provides methods for using amorphous form, Form D, Form E or Form F of varenicline tartrate as a medicament for the prevention or treatment of inflammatory bowel disease (including but not limited to ulcerative colitis, pyoderma gangrenosum and Crohn's disease), irritable bowel syndrome, spastic dystonia, chronic pain, acute pain, celiac sprue, pouchitis, vasoconstriction, anxiety, panic disorder, depression, bipolar disorder, autism, sleep disorders, jet lag, amyotrophic lateral sclerosis (ALS), cognitive dysfunction, hypertension, bulimia, anorexia, obesity, cardiac arrhythmias, gastric acid hypersecretion, ulcers, pheochromocytoma, progressive supranuclear palsy, chemical dependencies and addictions (e.g., dependencies on, or addictions to nicotine (and/or tobacco products), alcohol, benzodiazepines, barbiturates, opioids or cocaine), headache, migraine, stroke, traumatic brain injury (TBI), obsessive-compulsive disorder (OCD), psychosis, Huntington's chorea, tardive dyskinesia, hyperkinesia, dyslexia, schizophrenia, multi-infarct dementia, age-related cognitive decline, epilepsy, including petit mal absence epilepsy, senile dementia of the Alzheimer's type (AD), Parkinson's disease (PD), attention deficit hyperactivity disorder (ADHD) and Tourette's Syndrome, particularly, nicotine dependency, addition and withdrawal; including use in smoking cessation therapy.

### BRIEF DESCRIPTION OF THE ACCOMPANYING DRAWINGS

FIG. 1: X-ray powder diffraction (X-RPD) pattern of Form B of varenicline tartrate.

FIG. 2: X-ray powder diffraction (X-RPD) pattern of Form C of varenicline tartrate.

FIG. 3: X-ray powder diffraction (X-RPD) pattern of Form D of varenicline tartrate prepared from Example 3.

FIG. 4: X-ray powder diffraction (X-RPD) pattern of Form D of varenicline tartrate prepared from Example 4.

FIG. 5: X-ray powder diffraction (X-RPD) pattern of Form E of varenicline tartrate.

FIG. 6: X-ray powder diffraction (X-RPD) pattern of Form F of varenicline tartrate.

FIG. 7: X-ray powder diffraction (X-RPD) pattern of Form C of varenicline tartrate.

FIG. 8: Differential scanning calorimetry (DSC) of Form B of varenicline tartrate.

FIG. 9: Differential scanning calorimetry (DSC) of Form C of varenicline tartrate.

FIG. 10: Differential scanning calorimetry (DSC) of Form D of varenicline tartrate prepared from Example 3.

FIG. 11: Differential scanning calorimetry (DSC) of Form D of varenicline tartrate prepared from Example 4.

FIG. 12: Differential scanning calorimetry (DSC) of Form E of varenicline tartrate.

FIG. 13: Differential scanning calorimetry (DSC) of Form F of varenicline tartrate.

FIG. 14: Thermogravimetric analysis (TGA) of Form B of varenicline tartrate.

FIG. 15: Thermogravimetric analysis (TGA) of Form C of varenicline tartrate.

FIG. 16: Thermogravimetric analysis (TGA) of Form D of varenicline tartrate prepared from Example 3.

FIG. 17: Thermogravimetric analysis (TGA) of Form D of varenicline tartrate prepared from Example 4.

FIG. 18: Thermogravimetric analysis (TGA) of Form E of varenicline tartrate.

FIG. 19: Thermogravimetric analysis (TGA) of Form F of varenicline tartrate.

FIG. 20: Fourier transform infrared (FT-IR) spectrum of Form B of varenicline tartrate.

FIG. 21: Fourier transform infrared (FT-IR) spectrum of Form C of varenicline tartrate.

FIG. 22: Fourier transform infrared (FT-IR) spectrum of Form D of varenicline tartrate from Example 3.

FIG. 23: Fourier transform infrared (FT-IR) spectrum of Form D of varenicline tartrate from Example 4.

FIG. 24: Fourier transform infrared (FT-IR) spectrum of Form E of varenicline tartrate.

FIG. 25: Fourier transform infrared (FT-IR) spectrum of Form F of varenicline tartrate.

FIG. 26: X-ray powder diffraction pattern of amorphous form of varenicline tartrate.

### DETAILED DESCRIPTION OF THE INVENTION

Unless otherwise indicated, the following definitions are set forth to illustrate and define the meaning and scope of the various terms used to describe the invention herein.

The term “polymorphic form, polymorph, polymorph form or crystalline polymorph of varenicline tartrate” in the present invention refers to a crystal modification of varenicline tartrate, which can be characterized by analytical methods such as X-ray powder diffraction pattern, thermogravimetric analysis (TGA), differential scanning calorimetry (DSC), Fourier transform infrared spectroscopy (FT-IR), by its melting point or other techniques.

The term “pharmaceutically acceptable” means that which is useful in preparing a pharmaceutical composition that is generally non-toxic and is not biologically undesirable and includes that which is acceptable for veterinary use and/or human pharmaceutical use.

The term “pharmaceutical composition” or “pharmaceutical formulation” is intended to encompass a drug product including the active ingredient(s), pharmaceutically acceptable excipients that make up the carrier, as well as any product which results, directly or indirectly, from combination, complexation or aggregation of any two or more of the ingredients. Accordingly, the pharmaceutical compositions of the present invention encompass any composition made by admixing the active ingredient, active ingredient dispersion or composite, additional active ingredient(s), and pharmaceutically acceptable excipients.

The term “composition” is intended to encompass a particular pure polymorphic (or phase pure) form or a mixture of a particular polymorphic form along with other polymorph forms, solvate, amorphous form, hydrate or co-crystals. The composition may comprise a particular polymorphic form from a trace amount or less than 0.1% to 100% (weight by weight) based on the total amount of varenicline tartrate in the composition.

The term “about” generally means within 15%, preferably within 5%, and more preferably within 1% of a given value or range. Alternatively, the term “about” means within an acceptable standard error of the mean, when considered by one of ordinary skill in the art.

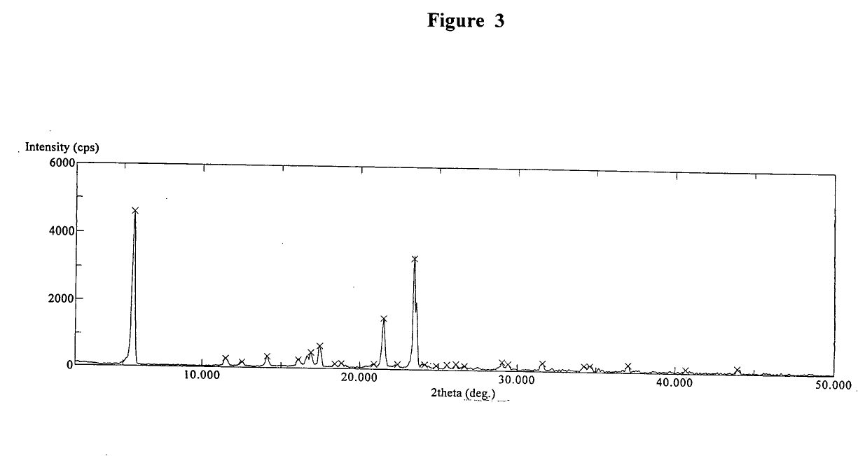

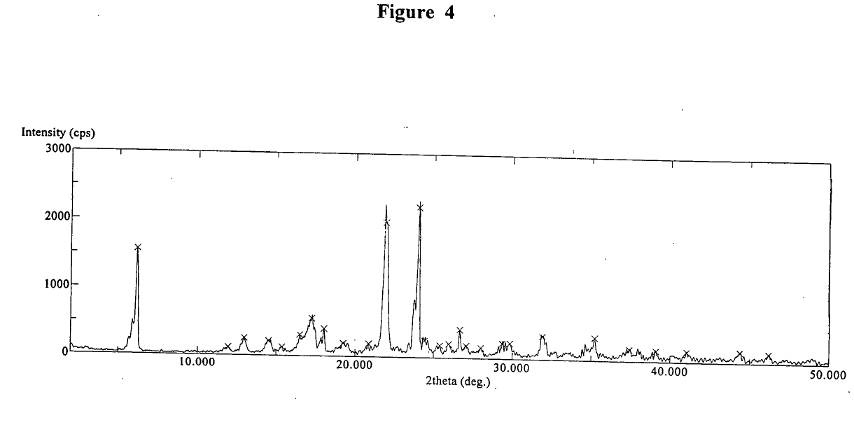

According to one aspect of the present invention, there is provided a novel polymorphic form of L-tartrate salt of 5,8,14-triazatetracyclo[10.3.1.0^{2,11}.0^{4,9}]-hexadeca-2(11),3,5,7,9-pentaene (varenicline tartrate or Compound I), designated as Form D herein, having an X-ray powder diffraction pattern (X-RPD), or substantially the same X-ray powder diffraction pattern, as shown in FIG. 3 or FIG. 4. More particularly, polymorphic Form D of varenicline tartrate according to the present invention can be characterized as having an X-ray diffraction pattern with characteristic peaks (expressed in 2θ±0.2° 2θ) at one or more of the following positions: 5.66, 11.48, 16.10, 16.90, 17.46, 18.42, 21.50 and 23.42. Form D of varenicline tartrate according to the present invention can be further characterized as having an X-ray diffraction pattern with characteristic peaks (expressed in 2θ±0.2° 2θ) at one or more of the following positions: 5.66, 11.48, 12.52, 14.12, 16.10, 16.90, 17.46, 18.42, 18.84, 20.90, 21.50, 22.40, 23.42, 24.12, 24.86, 25.54, 26.12, 26.66, 29.04, 29.42, 31.60, 34.24, 34.62, 37.04, 40.66 or 43.94.

Characterizing data for Form D of varenicline tartrate according to the present invention as obtained by X-ray powder diffraction is substantially the same as shown in FIG. 3 or FIG. 4 and Table 1.

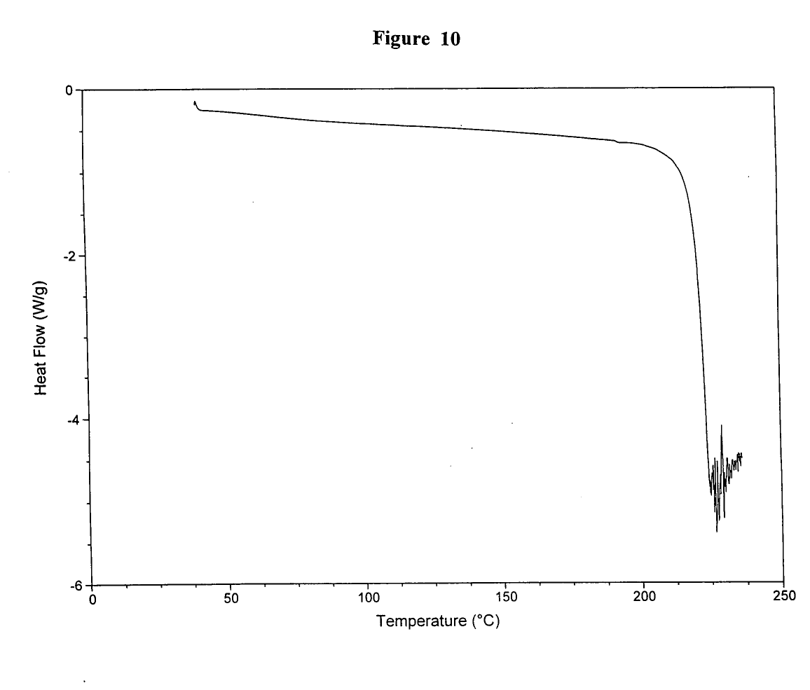

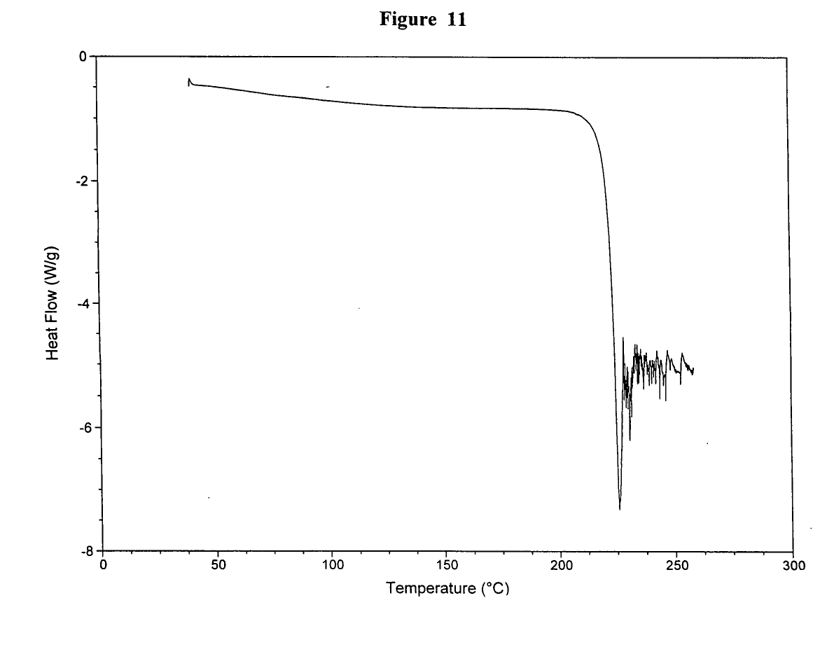

Further characterizing data for Form D of varenicline tartrate according to the present invention as obtained by differential scanning calorimetry (DSC) is substantially the same as shown in FIG. 10 or FIG. 11, and it provides an endothermic peak at around 223-228° C. (typically about 225-226° C.).

TABLE 1
Characteristic X-ray Powder Diffraction Pattern Peaks (expressed
in 2θ ± 0.2° 2θ) and Relative Intensities of Diffraction
Lines for Form D of Varenicline Tartrate

| Degree 2θ ± 0.2° 2θ | I/Iₒ |
|---|---|
| 5.66 | 100 |
| 11.48 | 6 |
| 12.52 | 4 |
| 14.12 | 8 |
| 16.10 | 6 |
| 16.90 | 11 |
| 17.46 | 15 |
| 18.42 | 4 |
| 18.84 | 4 |
| 20.90 | 4 |
| 21.50 | 34 |
| 22.40 | 4 |
| 23.42 | 73 |
| 24.12 | 4 |
| 24.86 | 3 |
| 25.54 | 4 |
| 26.12 | 4 |
| 26.66 | 3 |
| 29.04 | 6 |
| 29.42 | 5 |
| 31.60 | 6 |
| 34.24 | 4 |
| 34.62 | 4 |
| 37.04 | 5 |
| 40.66 | 3 |
| 43.94 | 4 |

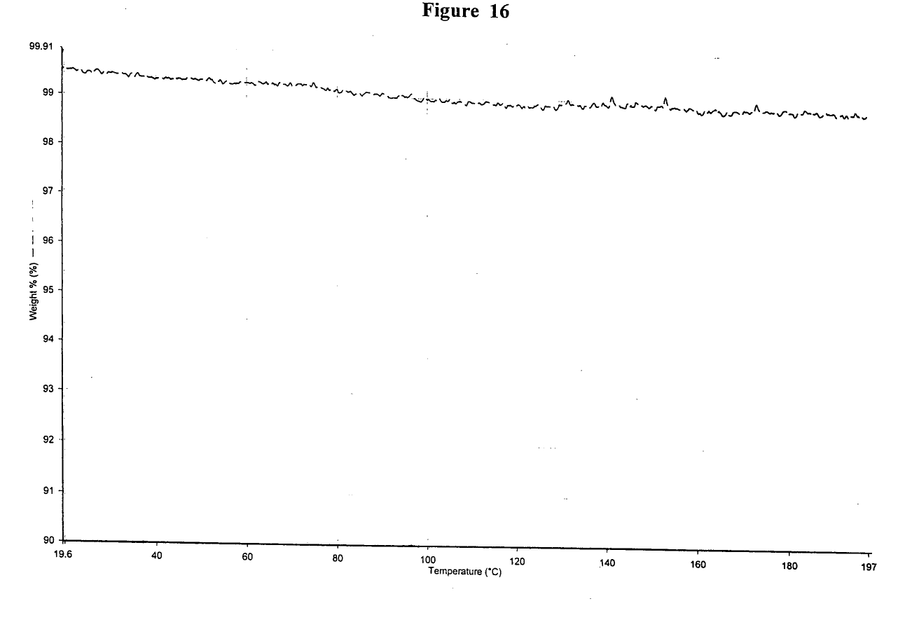

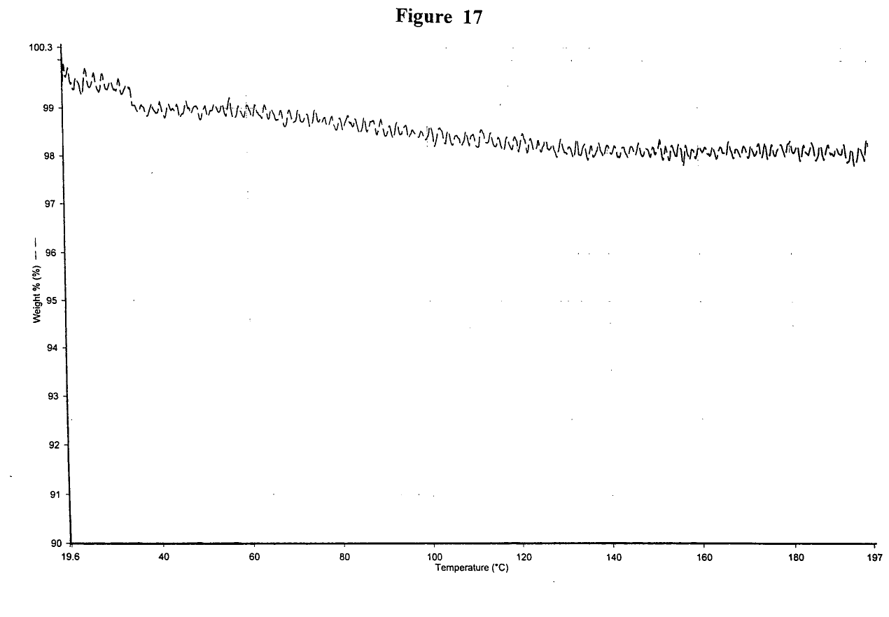

Still further characterizing data for polymorphic Form D of varenicline tartrate according to the present invention obtained by thermogravimetric analysis (TGA) is substantially the same as shown in FIG. 16 or FIG. 17, and they provide a loss of water at less than or about 0.3% w/w from 60° C. to 160° C. for Form D obtained in methanol and water (FIG. 16) or a loss of water or DMF or their mixtures at less than or about 0.8% w/w from 60° C. to 160° C. for Form D obtained in DMF and water (FIG. 17). Without binding any theory, the applicant thinks that the small amount of water residue or solvent DMF residue present in the product may not be a part of crystalline varenicline tartrate structure, and it may be free water residue or free DMF solvent residue, which can be removed by vacuum drying. The varenicline tartrate monohydrate would theoretically provide a loss of water at about 5.0% w/w and a semi-hydrate of varenicline tartrate would theoretically provide a loss of water at about 2.5% w/w. Therefore, Form D of varenicline tartrate according to the present invention is an anhydrous crystalline varenicline tartrate. The anhydrous nature of Form D of varenicline tartrate is further confirmed by lack of an endothermic peak at around 70-90° C. in its DSC. Anhydrous Form A or anhydrous Form B of varenicline tartrate disclosed in U. S. Pat. No. 6,890,927 or U.S. Pat. No. 7,265,119 also does not have the corresponding endothermic peak at about 70-90° C. in its DSC, which is a characteristic peak for crystalline varenicline tartrate hydrate (e.g., Form C).

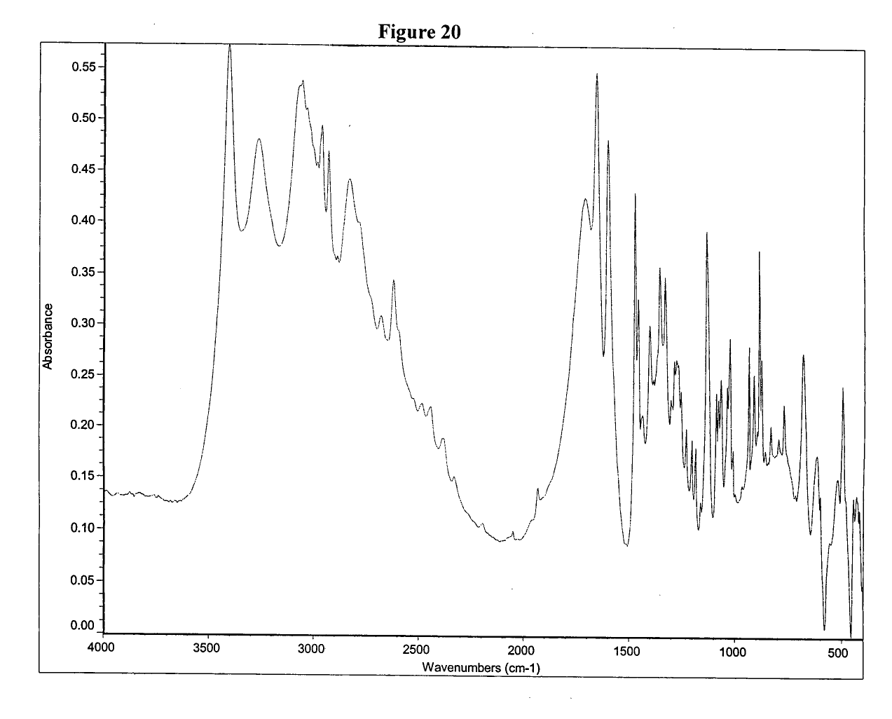

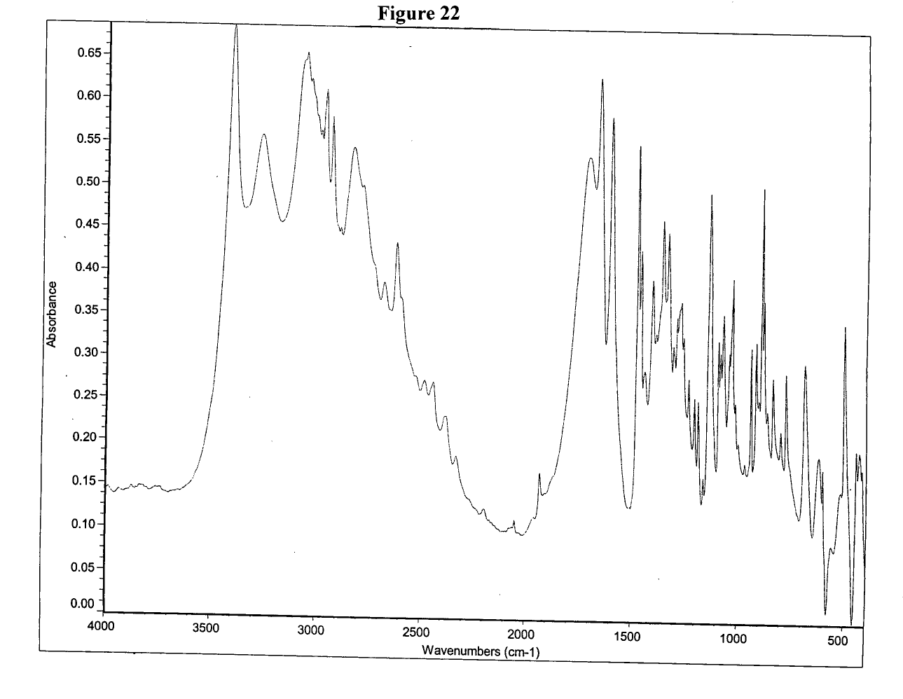

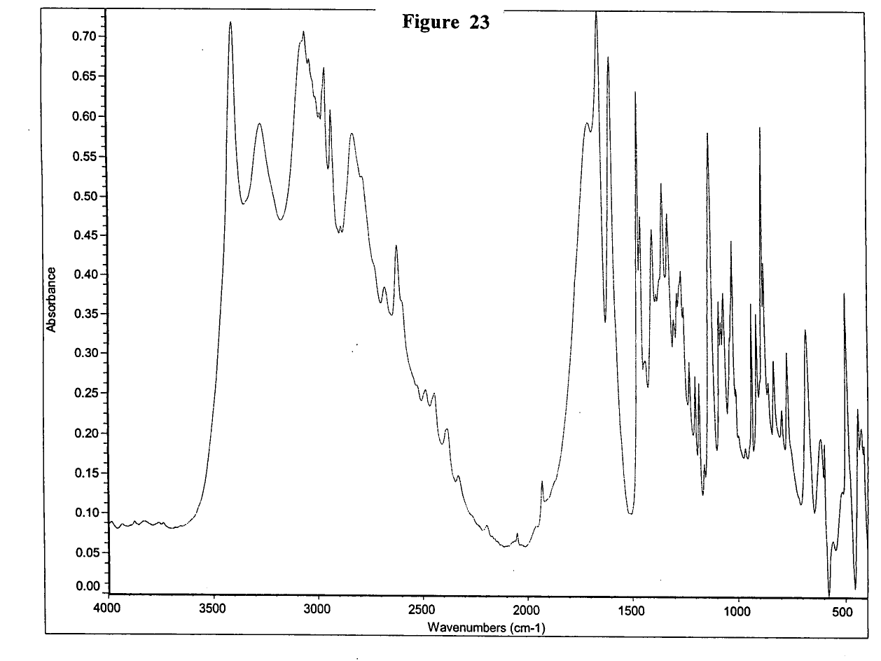

Further characterizing data for polymorphic Form D of varenicline tartrate according to the present invention obtained by the Fourier transform infrared (FT-IR) spectrum is substantially the same as shown in FIG. 22 or FIG. 23, and they contain peaks at one or more of the following positions of about 3402, 3264, 3054, 2960, 2929, 2828, 2619, 1706, 1654, 1601, 1472, 1458, 1403, 1356, 1331, 1303, 1268, 1231, 1203, 1185, 1135, 1090, 1081, 1068, 1026, 938, 914, 889, 879, 835, 799, 774, 685 or 501 cm⁻¹. FT-IR spectrum of Form D displays a characteristic feature at around 1300-1260 cm⁻¹, which is clearly different from FT-IR spectrum of Form B in the same region as shown in FIG. 20.

The crystalline Form D of anhydrous varenicline tartrate according to the present invention is thermally stable. Additionally, the applicant discovered that Form D can be prepared by recrystallizing varenicline tartrate in aqueous solvents and that Form D is not hygroscropic. Furthermore, Form D does not undergo a phase transformation even heating up to 40° C. Form D has good material flow characteristic and adequate chemical stability. These favorable characters render Form D of varenicline tartrate a superior polymorphic form for pharmaceutical formulation and bulk handling. The anhydrous Form A and Form B disclosed in reference are prepared from organic solvents only. As disclosed in U.S. Pat. No. 7,265,119 (column 9, line 50-65), Form A is the kinetic polymorph, not thermally favorable and it converts into Form B under appropriate conditions.

In one favored aspect, the polymorph Form D of varenicline tartrate provides X-ray powder diffraction (X-RPD) pattern substantially in accordance with FIG. 3, FIG. 4 or Table 1.

In one favored aspect, the Form D of varenicline tartrate provides differential scanning calorimetry (DSC) substantially in accordance with FIG. 10 or FIG. 11.

In one still favored aspect, the Form D of varenicline tartrate provides thermogravimetric analysis (TGA) substantially in accordance with FIG. 16 or FIG. 17.

In one favored aspect, the Form D of varenicline tartrate provides the Fourier transform infrared (FT-IR) substantially in accordance with FIG. 22 or FIG. 23.

The present invention encompasses Form D of varenicline tartrate isolated in pure form or in a mixture as a solid composition when admixed with other materials, for example the other known polymorphic forms (i.e. amorphous form, solvates, Form A, Form B, Form C, Form E, Form F or other forms) of varenicline tartrate or any other materials.

Thus in one aspect there is provided Form D of crystalline varenicline tartrate in isolated solid form.

In a further aspect there is provided Form D of varenicline tartrate in phase pure form. The phase pure form means that Form D is over 95% (w/w), preferably over 98% (w/w), more preferably over 99% (w/w) and most preferably over 99.5% (w/w) or over 99.9% (w/w).

Specifically, the present invention provides that Form D of varenicline tartrate is in the form of a composition or a mixture of Form D along with one or more other crystalline, solvate, amorphous, or other polymorphic forms or their combinations thereof of varenicline tartrate. Such a composition may be a drug substance or an active ingredient or a dispersion in pharmaceutical compositions or formulations. For example, such composition may comprise polymorphic Form D along with one or more other polymorphic forms of varenicline tartrate, such as amorphous form, hydrate, solvates, polymorph Form A, Form B, Form C, Form E, Form F and/or other forms or their combinations thereof. More specifically, the composition may comprise from trace amounts up to 100% Form D, or any amount in between-for example, the composition may comprise less than 0.1%, 0.5%, 1%, 2%, 5%, 10%, 20%, 30%,40% or 50% by weight of Form D based on the total amount of varenicline tartrate in the composition. Alternatively, the composition may comprise at least 50%, 60%, 70%, 80%, 90%, 95%, 97%, 98%, 99%, 99.5% or 99.9% by weight of Form D based on the total amount of varenicline tartrate in the composition.

In yet a further aspect there is provided Form D of varenicline tartrate in crystalline form.

In a preferred aspect, the particle size of polymorphic Form D of varenicline tartrate in the present invention has the median value of the volume mean diameter of the particles within the range of 0.01 μm-450 μm, preferably 5-250 μm, and most preferably 50-150 μm. Such particles are better in good material flow characteristics, improving the uniformity of dosage forms and thus suitable or superior for bulk preparation and formulation advantages.

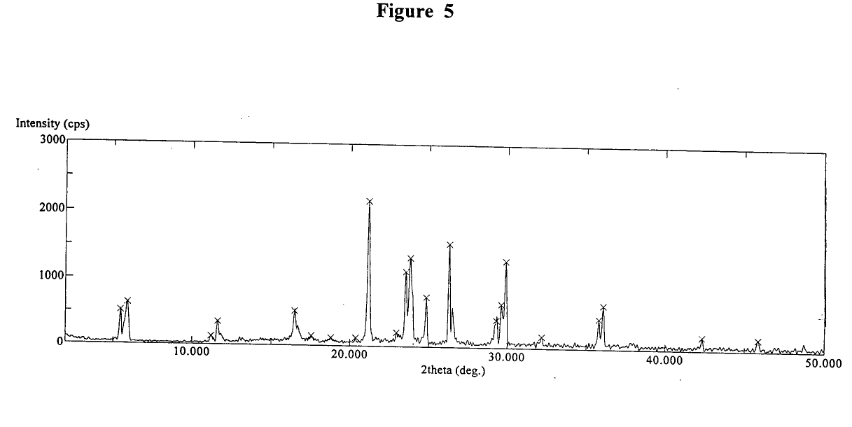

According to another aspect of the present invention, there is provided a novel polymorphic form of L-tartrate salt of 5,8,14-triazatetracyclo[10.3.1.0^{2,11}.0^{4,9}]-hexadeca-2(11),3,5,7,9-pentaene (varenicline tartrate or Compound I), designated as Form E herein, having an X-ray powder diffraction pattern (X-RPD), or substantially the same X-ray powder diffraction pattern, as shown in FIG. 5. More particularly, polymorphic Form E of varenicline tartrate according to the present invention can be characterized as having an X-ray diffraction pattern with characteristic peaks (expressed in 2θ±0.2° 2θ) at one or more of the following positions: 5.44, 5.88, 11.62, 16.48, 21.16, 23.54, 23.82, 24.84, 26.30, 29.30, 29.62, 29.90, 35.80 and 36.08. Form E of varenicline tartrate according to the present invention can be further characterized as having an X-ray diffraction pattern with characteristic peaks (expressed in 2θ±0.2° 2θ) at one or more of the following positions: 5.44, 5.88, 11.20, 11.62, 16.48, 20.36, 21.16, 22.96, 23.54, 23.82, 24.84, 26.30, 29.30, 29.62, 29.90, 32.18, 35.80, 36.08, 42.32 or 45.86.

Characterizing data for Form E of varenicline tartrate according to the present invention as obtained by X-ray powder diffraction is substantially the same as shown in FIG. 5 and Table 2.

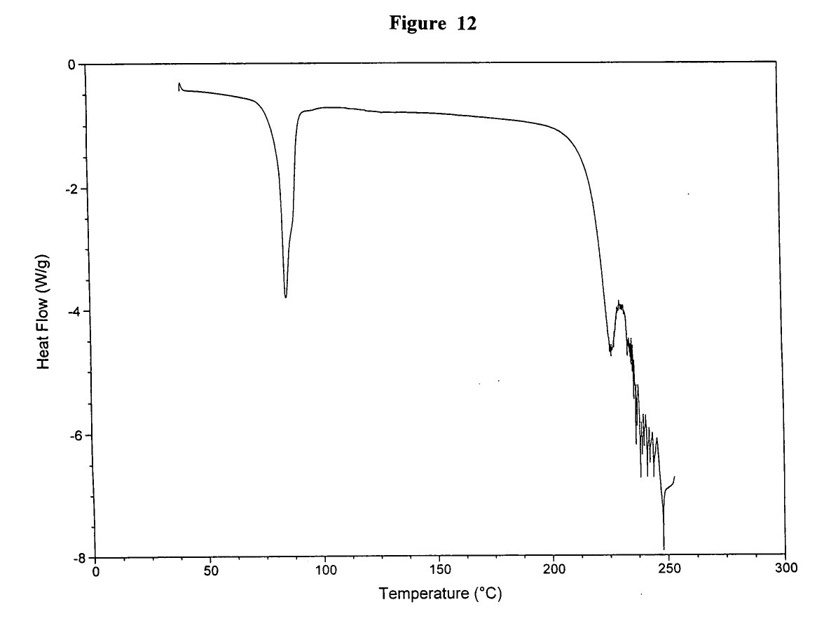

Further characterizing data for Form E of varenicline tartrate according to the present invention as obtained by differential scanning calorimetry (DSC) is substantially the same as shown in FIG. 12, and it provides an endothermic peak at about 82-88° C. (typically about 84-86° C.) and another endothermic peak at about 224-228° C. (typically about 225-226° C.).

TABLE 2
Characteristic X-ray Powder Diffraction Pattern Peaks
(expressed in 2θ ± 0.2° 2θ) and Relative Intensities of
Diffraction Lines for Form E of Varenicline Tartrate

| Degree 2θ ± 0.2° 2θ | I/Iₒ |
|---|---|
| 5.44 | 24 |
| 5.88 | 30 |
| 11.20 | 7 |
| 11.62 | 17 |
| 16.48 | 25 |
| 20.36 | 7 |
| 21.16 | 100 |
| 22.96 | 11 |
| 23.54 | 53 |
| 23.82 | 62 |
| 24.84 | 35 |
| 26.30 | 72 |
| 29.30 | 20 |
| 29.62 | 31 |
| 29.90 | 61 |
| 32.18 | 9 |
| 35.80 | 22 |
| 36.08 | 31 |
| 42.32 | 10 |
| 45.86 | 9 |

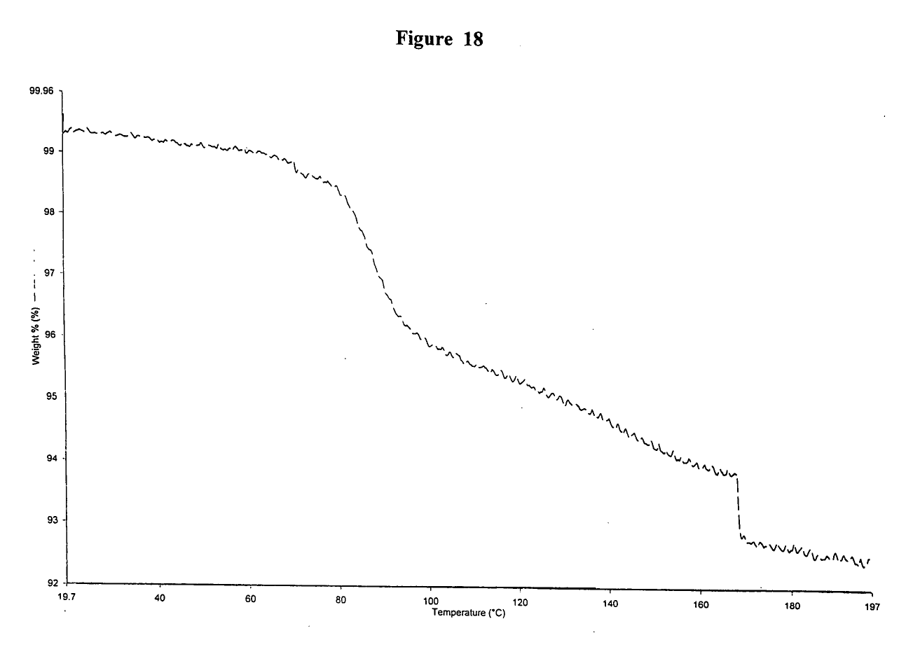

Still further characterizing data for polymorphic Form E of varenicline tartrate according to the present invention obtained by thermogravimetric analysis (TGA) is substantially the same as shown in FIG. 18, and it provides a loss of water at about 5.0% w/w from 60° C. to 160° C. The varenicline tartrate monohydrate would theoretically provide a loss of water at about 5.0% w/w and a semi-hydrate of varenicline tartrate would theoretically provide a loss of water at about 2.5% w/w. Therefore, Form E of varenicline tartrate according to the present invention is a crystalline varenicline tartrate monohydrate, which is further confirmed by an endothermic peak at around 84-86° C. in its DSC as shown in FIG. 12. Form B disclosed in U.S. Pat. No. 7,265,119, or Form D in the current invention are anhydrous varenicline tartrate and thus they do not have the corresponding endothermic peak at about 70-90° C. in its DSC. Form C is varenicline tartrate monohydrate and thus displays a characteristic endothermic peak at about 76-78° C. Therefore, both X-RPD and DSC profiles of Form E are different from those of other known polymorphic forms, including Form C. Therefore, Form E according to the present invention is a new and novel crystalline varenicline tartrate monohydrate.

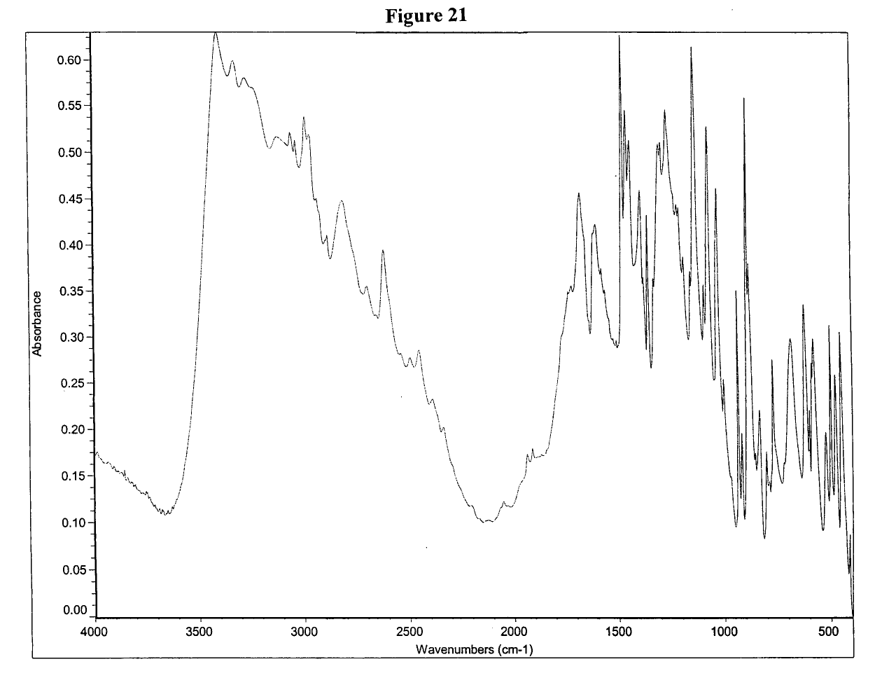

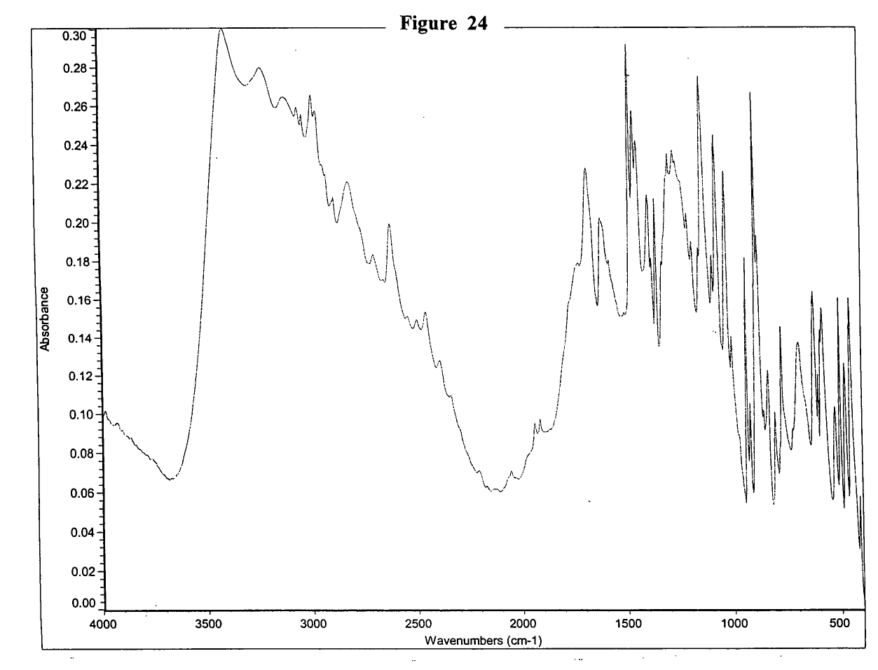

Further characterizing data for polymorphic Form E of varenicline tartrate according to the present invention obtained by the Fourier transform infrared (FT-IR) spectrum is substantially the same as shown in FIG. 24, and it contains peaks at one or more of the following positions of about 3402, 2984, 2813, 2616, 1676, 1614, 1475, 1455, 1438, 1389, 1356, 1291, 1266, 1136, 1090, 1071, 1027, 1002, 936, 919, 890, 879, 834, 804, 772, 691, 621, 600, 580, 524, 501, 478 or 452 cm⁻¹. FT-IR spectrum of Form E displays characteristic bands at around 1300-1200 cm⁻¹, which is clearly different from FT-IR spectrum of Form C in the same region as shown in FIG. 21. Therefore FT-IR data also confirm that Form E is new crystalline varenicline tartrate monohydrate.

In one favored aspect, the polymorph Form E of varenicline tartrate provides X-ray powder diffraction (X-RPD) pattern substantially in accordance with FIG. 5 and Table 2.

In one favored aspect, the Form E of varenicline tartrate provides differential scanning calorimetry (DSC) substantially in accordance with FIG. 12.

In one still favored aspect, the Form E of varenicline tartrate provides thermogravimetric analysis (TGA) substantially in accordance with FIG. 18.

In one favored aspect, the Form E of varenicline tartrate provides the Fourier transform infrared (FT-IR) substantially in accordance with FIG. 24.

The present invention encompasses Form E of varenicline tartrate isolated in pure form or in a mixture as a solid composition when admixed with other materials, for example the other known polymorphic forms (i.e. amorphous form, solvates, Form A, Form B, Form C, Form D, Form F or other forms) of varenicline tartrate or any other materials.

Thus in one aspect there is provided Form E of crystalline varenicline tartrate in isolated solid form.

In a further aspect there is provided Form E of varenicline tartrate in phase pure form. The phase pure form means that Form E is over 95% (w/w), preferably over 98% (w/w), more preferably over 99% (w/w) and most preferably over 99.5% (w/w) or over 99.9% (w/w).

Specifically, the present invention provides that Form E of varenicline tartrate is in the form of a composition or a mixture of Form E along with one or more other crystalline, solvate, amorphous, or other polymorphic forms or their combinations thereof of varenicline tartrate. Such a composition may be a drug substance or an active ingredient or a dispersion in pharmaceutical compositions or formulations. For example, such composition may comprise polymorphic Form E along with one or more other polymorphic forms of varenicline tartrate, such as amorphous form, hydrate, solvates, polymorph Form A, Form B, Form C, Form D, Form F and/or other forms or their combinations thereof. More specifically, the composition may comprise from trace amounts up to 100% Form E, or any amount in between-for example, the composition may comprise less than 0.1%, 0.5%, 1%, 2%, 5%, 10%, 20%, 30%, 40% or 50% by weight of Form E based on the total amount of varenicline tartrate in the composition. Alternatively, the composition may comprise at least 50%, 60%, 70%, 80%, 90%, 95%, 97%, 98%, 99%, 99.5% or 99.9% by weight of Form E based on the total amount of varenicline tartrate in the composition.

In yet a further aspect there is provided Form E of varenicline tartrate in crystalline form.

In a preferred aspect, the particle size of polymorphic Form E of varenicline tartrate in the present invention has the median value of the volume mean diameter of the particles within the range of 0.01 μm-450 μm, preferably 5-250 μm, and most preferably 50-150 μm. Such particles are better in good material flow characteristics, improving the uniformity of dosage forms and thus suitable or superior for bulk preparation and formulation advantages.

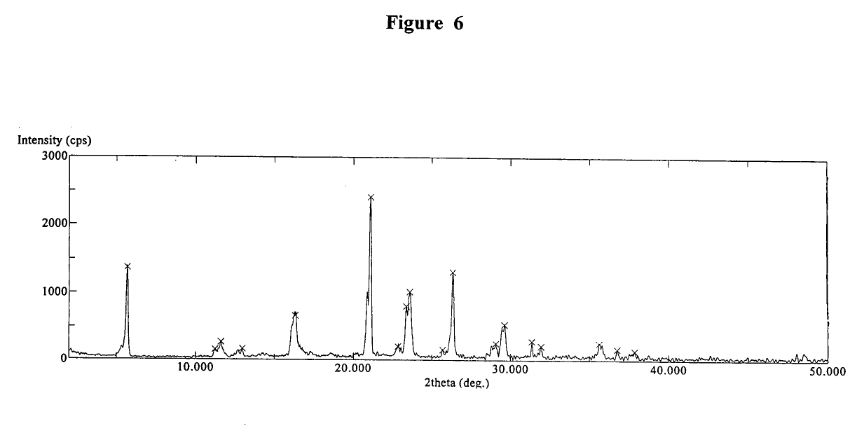

According to another aspect of the present invention, there is provided a novel polymorphic form of L-tartrate salt of 5,8,14-triazatetracyclo[10.3.1.0^{2,11}.0^{4,19}]-hexadeca-2(11),3,5,7,9-pentaene (varenicline tartrate or Compound I), designated as Form F herein, having an X-ray powder diffraction pattern (X-RPD), or substantially the same X-ray powder diffraction pattern, as shown in FIG. 6. More particularly, polymorphic Form F of varenicline tartrate according to the present invention can be characterized as having an X-ray diffraction pattern with characteristic peaks (expressed in 2θ±0.2° 2θ) at one or more of the following positions: 5.68, 11.60, 16.32, 21.08, 23.38, 23.60, 26.34, 29.62 and 31.94. Form F of varenicline tartrate according to the present invention can be further characterized as having an X-ray diffraction pattern with characteristic peaks (expressed in 2θ±0.2° 2θ) at one or more of the following positions: 5.68, 11.22, 11.60, 12.96, 16.32, 21.08, 22.84, 23.38, 23.60, 25.70, 26.34, 29.06, 29.62, 31.36, 31.94, 35.62, 36.74 or 37.84.

Characterizing data for Form F of varenicline tartrate according to the present invention as obtained by X-ray powder diffraction is substantially the same as shown in FIG. 6 and Table 3.

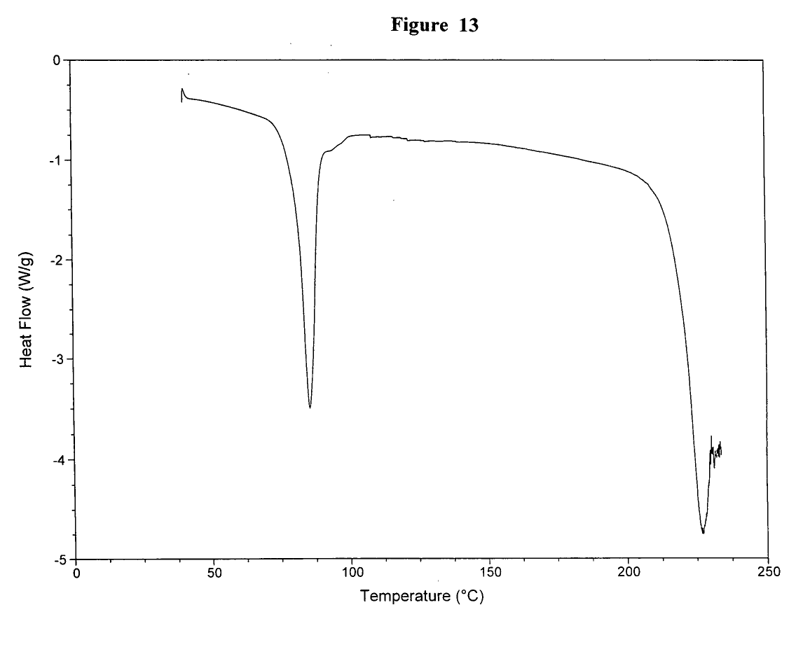

Further characterizing data for Form F of varenicline tartrate according to the present invention as obtained by differential scanning calorimetry (DSC) is substantially the same as shown in FIG. 13, and it provides an endothermic peak at about 82-88° C. (typically about 85-86° C.) and another endothermic peak at about 224-227° C. (typically about 225-226° C.).

TABLE 3
Characteristic X-ray Powder Diffraction Pattern Peaks
(expressed in 2θ ± 0.2° 2θ) and Relative Intensities of
Diffraction Lines for Form F of Varenicline Tartrate

| Degree 2θ ± 0.2 ° 2θ | I/Iₒ |
|---|---|
| 5.68 | 58 |
| 11.22 | 7 |
| 11.60 | 12 |
| 12.96 | 7 |
| 16.32 | 28 |
| 21.08 | 100 |
| 22.84 | 9 |
| 23.38 | 34 |
| 23.60 | 43 |
| 25.70 | 7 |
| 26.34 | 55 |
| 29.06 | 11 |
| 29.62 | 23 |
| 31.36 | 12 |
| 31.94 | 9 |
| 35.62 | 11 |
| 36.74 | 8 |
| 37.84 | 6 |

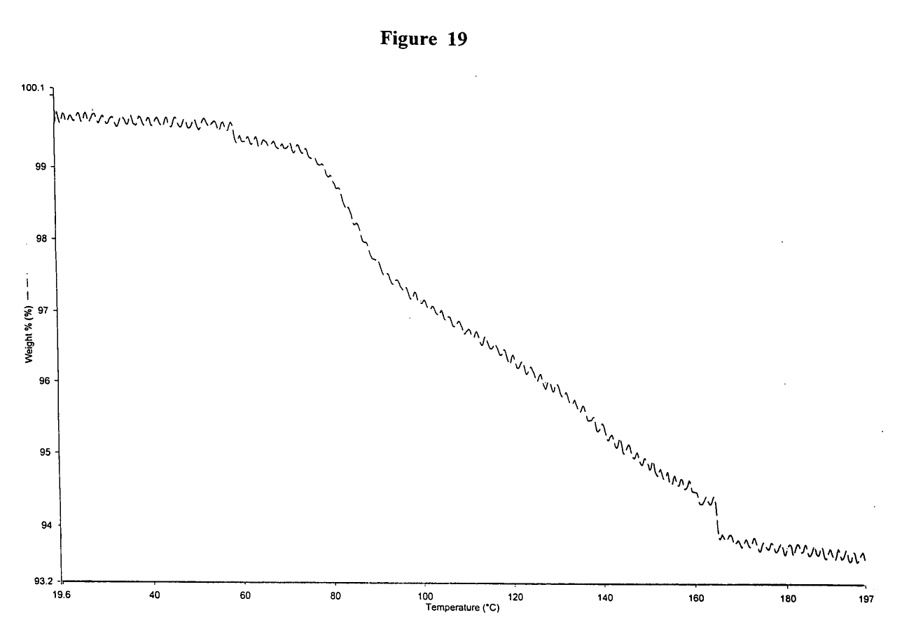

Still further characterizing data for polymorphic Form F of varenicline tartrate according to the present invention obtained by thermogravimetric analysis (TGA) is substantially the same as shown in FIG. 19, and it provides a loss of water at about 5.0% w/w from 60° C. to 160° C. The varenicline tartrate monohydrate would theoretically provide a loss of water at about 5.0% w/w and a semi-hydrate of varenicline tartrate would theoretically provide a loss of water at about 2.5% w/w. Therefore, Form F of varenicline tartrate according to the present invention is a crystalline varenicline tartrate monohydrate, which is further confirmed by an endothermic peak at around 85-86° C. in its DSC as shown in FIG. 13. Form B disclosed in U.S. Pat. No. 7,265,119, or Form D in the current invention are anhydrous varenicline tartrate and thus they do not have the corresponding endothermic peak at about 70-90° C. in its DSC. Form C is varenicline tartrate monohydrate and thus displays a characteristic endothermic peak at about 76-78° C. Therefore, both X-RDP and DSC profiles of Form F are different from those of other known polymorphic forms, including Form C or Form E. Therefore, Form F according to the present invention is another new and novel crystalline varenicline tartrate monohydrate.

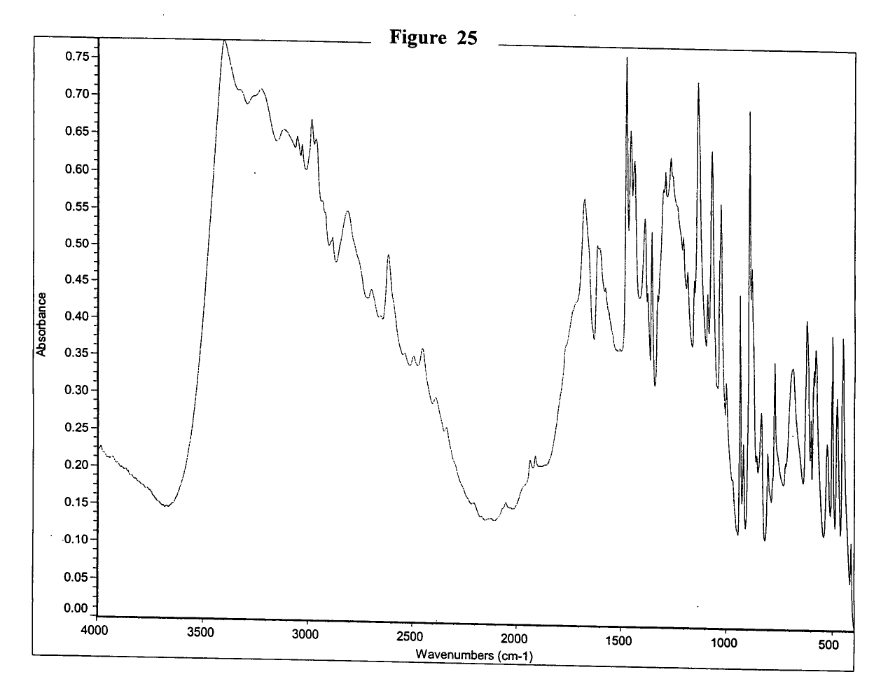

Further characterizing data for polymorphic Form F of varenicline tartrate according to the present invention obtained by the Fourier transform infrared (FT-IR) spectrum is substantially the same as shown in FIG. 25, and it contains peaks at one or more of the following positions of about 3402, 2984, 2811, 2616, 1676, 1614, 1475, 1455, 1438, 1389, 1356, 1291, 1266, 1136, 1090, 1071, 1027, 1002, 936, 919, 890, 880, 835, 804, 772, 689, 621, 601, 580, 524, 501, 478 or 453 cm⁻¹. FT-IR spectrum of Form F displays characteristic bands at around 1300-1200 cm⁻¹, which is clearly different from FT-IR spectrum of Form C in the same region as shown in FIG. 21. Therefore FT-IR data also confirm that Form F is new crystalline varenicline tartrate monohydrate.

In one favored aspect, the polymorph Form F of varenicline tartrate provides X-ray powder diffraction (X-RPD) pattern substantially in accordance with FIG. 6 and Table 3.

In one favored aspect, the Form F of varenicline tartrate provides differential scanning calorimetry (DSC) substantially in accordance with FIG. 13.

In one still favored aspect, the Form F of varenicline tartrate provides thermogravimetric analysis (TGA) substantially in accordance with FIG. 19.

In one favored aspect, the Form F of varenicline tartrate provides the Fourier transform infrared (FT-IR) substantially in accordance with FIG. 25.

The present invention encompasses Form F of varenicline tartrate isolated in pure form or in a mixture as a solid composition when admixed with other materials, for example the other known polymorphic forms (i.e. amorphous form, solvates, Form A, Form B, Form C, Form D, Form E or other forms) of varenicline tartrate or any other materials.

Thus in one aspect there is provided Form F of crystalline varenicline tartrate in isolated solid form.

In a further aspect there is provided Form F of varenicline tartrate in phase pure form. The phase pure form means that Form F is over 95% (w/w), preferably over 98% (w/w), more preferably over 99% (w/w) and most preferably over 99.5% (w/w) or over 99.9% (wlw).

Specifically, the present invention provides that Form F of varenicline tartrate is in the form of a composition or a mixture of Form F along with one or more other crystalline, solvate, amorphous, or other polymorphic forms or their combinations thereof of varenicline tartrate. Such a composition may be a drug substance or an active ingredient or a dispersion in pharmaceutical compositions or formulations. For example, such composition may comprise polymorphic Form F along with one or more other polymorphic forms of varenicline tartrate, such as amorphous form, hydrate, solvates, polymorph Form A, Form B, Form C, Form D, Form E and/or other forms or their combinations thereof. More specifically, the composition may comprise from trace amounts up to 100% Form F, or any amount in between-for example, the composition may comprise less than 0.1%, 0.5%, 1%, 2%, 5%, 10%, 20%, 30%, 40% or 50% by weight of Form E based on the total amount of varenicline tartrate in the composition. Alternatively, the composition may comprise at least 50%, 60%, 70%, 80%, 90%, 95%, 97%, 98%, 99%, 99.5% or 99.9% by weight of Form F based on the total amount of varenicline tartrate in the composition.

In yet a further aspect there is provided Form F of varenicline tartrate in crystalline form.

In a preferred aspect, the particle size of polymorphic Form F of varenicline tartrate in the present invention has the median value of the volume mean diameter of the particles within the range of 0.01 μm-450 μm, preferably 5-250 μm, and most preferably 50-150 μm. Such particles are better in good material flow characteristics, improving the uniformity of dosage forms and thus suitable or superior for bulk preparation and formulation advantages.

According to still another aspect, the present invention provides three novel polymorphic forms of varenicline tartrate, e.g., Form D, Form E or Form of L-tartrate salt of 5,8,14-triazatetracyclo [10.3.1.0^{2,11}.0^{4,9}]-hexadeca-2(11),3,5,7,9-pentaene (varenicline tartrate or Compound I). The polymorphic form of varenicline tartrate herein refers to a solid composition of varenicline tartrate or a drug substance of varenicline tartrate that comprises more than 0.1% Form D, Form E or Form F of varenicline tartrate, preferably comprises more than 50% Form D, Form E or Form F of varenicline tartrate, more preferably contains more than 95% Form D, Form E or Form F of varenicline tartrate or essentially pure Form D, Form E or Form F of crystalline varenicline tartrate.

More specifically, according to one embodiment, the present invention provides the Compound I drug substance that comprises the Form D, Form E or Form F in a detectable amount. By “drug substance” is meant the active pharmaceutical ingredient (API). The amount of the Form D, Form E or Form F in the drug substance can be quantified by the use of physical methods such as X-ray powder diffraction, solid-state fluorine-19 magic-angle spinning (MAS) nuclear magnetic resonance spectroscopy, solid-state carbon-13 cross-polarization magic-angle spinning (CPMAS) nuclear magnetic resonance spectroscopy, solid state Fourier-transform infrared spectroscopy, and Raman spectroscopy. A detectable amount is an amount that can be detected by such physical methods. The limits of detection of such methods are about 0.1-5%, and are anticipated to improve with technological advances. The remainder of the drug substance may additionally comprise various crystalline forms of Compound I and polymorphs and pseudopolymorphs thereof. In a class of this embodiment, about 1% to about 100% by weight of the Form D, Form E or Form F is present in the drug substance. In a second class of this embodiment, about 10% to about 100% by weight of the Form D, Form E or Form F is present in the drug substance. In a third class of this embodiment, about 25% to about 100% by weight of the Form D, Form E or Form F is present in the drug substance. In a fourth class of this embodiment, about 50% to about 100% by weight of the Form D, Form E or Form F is present in the drug substance. In a fifth class of this embodiment, about 75% to about 100% by weight of the Form D, Form E or Form F is present in the drug substance. In a sixth class of this embodiment, substantially all of the Compound I drug substance is the Form D, Form E or Form F, i.e., the Compound I drug substance is substantially phase pure Form D, Form E or Form F.

Varenicline tartrate or Compound I of the present invention is L-tartrate salt of 5,8,14-triazatetracyclo[10.3.1.0^{2,11}.0^{4,9}]-hexadeca-2(11),3,5,7,9-pentaene accordingly, the present invention is directed to novel polymorphic forms of L-tartrate salt of 5,8,14-riazatetracyclo[0.3.1.0^{2,11}.0^{4,9}]-hexadeca-2(11),3,5,7,9-pentaene.

According to another aspect, the present invention provides a process for preparing polymorph Form D of varenicline tartrate. Polymorph Form D may be prepared by crystallization from a crystallization solvent containing varenicline tartrate. As used herein, the term “crystallization solvent” means a solvent or combination of solvents from which varenicline tartrate is preferentially crystallized as polymorph Form D. Representative crystallization solvents for preparation of Form D include water, methanol, N,N-dimethylformamide (DMF) and combinations thereof. In a preferred aspect, the crystallization solvent comprises methanol, N,N-dimethylformamide or combinations thereof, to which water is gradually added.

In a preferred aspect, Form D of varenicline tartrate may be prepared by slurring starting material, crude or pure varenicline tartrate, anhydrate (e.g., Form B) or solvate, which can be obtained according to the procedures described in U.S. Pat. No. 6,890,927 or U.S. Pat. No. 7,265,119 with water, methanol or DMF or a mixture of them under heat. The varenicline tartrate is very soluble in water, but not soluble in methanol or DMF. The concentration of varenicline tartrate within the solution may range from about 0.1% by weight to the saturation point. This concentration will, of course, vary depending upon the temperature at which the co-solvent solution is held, with warmer temperatures generally allowing for the preparation of more concentrated solutions of varenicline tartrate. Preferably, the concentration (w/w %) of varenicline tartrate starting material in solution is about 0.5-15%, preferably about 1-10%, more preferably about 1.5-5%. The volume ratio of methanol or DMF to water is about 2:0.1-2, preferably about 1:0.2-1.5, more preferably about 1:0.5-1.0, most preferably about 1:0.6-0.8. Water is then added into the above suspension, and the mixture is heated, suitably to a temperature in the range of from about 45° C. to 85° C., such as about 50° C. to 75° C., for example about 70° C. until all solid materials are dissolved. The clear and hot solution is allowed to cool down to ambient temperature, and the cooled solution is kept at about −20° C.-30° C. for crystallization, preferably at about −10° C.-15° C., and more preferably at about 0-10° C., and most preferably at about 5° C. The crystal Form D of varenicline tartrate is formed over a period of one to six days, and the crystal Form D is recovered from the solvent by filtration. The obtained crystal Form D can be dried under a vacuum oven at about 20° C.-60° C., preferably at about 35° C.-50° C., more preferably at about 35° C.-45° C., and most preferably at 38-42° C. for about 10-40 hours to remove the solvent residues.

Once obtained, crystals of polymorph Form D may be used as the nucleating agent or “seed” crystals for subsequent crystallizations of polymorph Form D from the crystallization solvent. In one embodiment, the crystallization solvent is formed by dissolving varenicline tartrate in a hot mixture of water and methanol or DMF or other suitable crystallization solvents. The crystallization solvent is then seeded with crystals of polymorph Form D, cooled and filtered, resulting in polymorph Form D. Such seeding with crystals of polymorph Form D may take place at any time during the slurring process. Alternatively, seeding with crystals of polymorph Form D may take place prior to, or simultaneously with, addition of varenicline tartrate to the crystallization solvent.

Form D of crystalline varenicline tartrate as obtained above is characterized by X-ray powder diffraction pattern, substantially the same as shown in FIG. 3, FIG. 4 and Table 1.

Form D of varenicline tartrate as obtained above is characterized by differential scanning calorimetry (DSC), substantially the same as shown in FIG. 10 or FIG. 11.

The crystals of varenicline tartrate obtained from recrystallization in solvents as described in above processes may have different crystal habits (e.g., shape), water contents, surface area, bulk or tap density, or particle size, but they clearly still belonged to a new and novel polymorphic form (Form D) of varenicline tartrate, as it is characterized and confirmed by X-ray powder diffraction pattern, DSC thermogram, TGA and FT-IR spectroscopy techniques. The X-ray powder diffraction pattern of Form D is clearly different from those of other known forms such as Form A, Form B or Form C in the prior art, and also different from those of Form E or Form F in the present application.

According to a further aspect, the present invention provides a process for preparing polymorph Form E of varenicline tartrate. Polymorph Form E may be prepared by crystallization from a crystallization solvent containing varenicline tartrate. As used herein, the term “crystallization solvent” means a solvent or combination of solvents from which varenicline tartrate is preferentially crystallized as polymorph Form E. Representative crystallization solvents for preparation of Form E include water, isopropanol (isopropyl alcohol, IPA) and combinations thereof. In a preferred aspect, the crystallization solvent comprises isopropanol, to which water is gradually added.

In a preferred aspect, Form E of varenicline tartrate may be prepared by slurring starting material, crude or pure varenicline tartrate, anhydrate (e.g., Form B) or solvate, which can be obtained according to the procedures described in U.S. Pat. No. 6,890,927 or U.S. Pat. No. 7,265,119 with isopropanol or a mixture of isopropanol and water under heat. The varenicline tartrate is very soluble in water, but not soluble in isopropanol. The concentration of varenicline tartrate within the solution may range from about 0.1% by weight to the saturation point. This concentration will, of course, vary depending upon the temperature at which the co-solvent solution is held, with warmer temperatures generally allowing for the preparation of more concentrated solutions of varenicline tartrate. Preferably, the concentration (w/w %) of varenicline tartrate starting material in solution is about 0.5-15%, preferably about 1-10%, more preferably about 1.5-5%. The volume ratio of isopropanol to water is about 5:0.1-5, preferably about 5:0.2-3, more preferably about 5:0.5-1.5, most preferably about 5:0.8-1.2. Water is then added into the above suspension, and the mixture is heated, suitably to a temperature in the range of from about 45° C. to 85° C., such as about 50° C. to 75° C., for example about 70° C. until all solid materials are dissolved. The clear and hot solution is allowed to cool down to ambient temperature, and the cooled solution is kept at about −20° C.-30° C. for crystallization, preferably at about −10° C.-15° C., and more preferably at about 0-10° C., and most preferably at about 5° C. The crystal Form E of varenicline tartrate is formed over a period of one to six days, and the crystal Form E is recovered from the solvent by filtration. The obtained crystal From E can be dried under a vacuum oven at about 20° C.-60° C., preferably at about 35° C.-50° C., more preferably at about 35° C.-45° C., and most preferably at 38-42° C. for about 10-40 hours to remove the solvent residues.

Once obtained, crystals of polymorph Form E may be used as the nucleating agent or “seed” crystals for subsequent crystallizations of polymorph Form E from the crystallization solvent. In one embodiment, the crystallization solvent is formed by dissolving varenicline tartrate in a hot mixture of isopropanol and water or other suitable crystallization solvents. The crystallization solvent is then seeded with crystals of polymorph Form E, cooled and filtered, resulting in polymorph Form E. Such seeding with crystals of polymorph Form E may take place at any time during the slurring process. Alternatively, seeding with crystals of polymorph Form E may take place prior to, or simultaneously with, addition of varenicline tartrate to the crystallization solvent.

Form E of crystalline varenicline tartrate as obtained above is characterized by X-ray powder diffraction pattern, substantially the same as shown in FIG. 5 and Table 2.

Form E of varenicline tartrate as obtained above is characterized by differential scanning calorimetry (DSC), substantially the same as shown in FIG. 12.

The crystals of varenicline tartrate obtained from recrystallization in solvents as described in above processes may have different crystal habits (e.g., shape), water contents, surface area, bulk or tap density, or particle size, but they clearly still belonged to a new and novel polymorphic form (Form E) of varenicline tartrate, as it is characterized and confirmed by X-ray powder diffraction pattern, DSC thermogram, TGA and FT-IR spectroscopy techniques. The X-ray powder diffraction pattern of Form E is clearly different from those of other known forms such as Form A, Form B or Form C in the prior art, and also different from those of Form D or Form F in the present application.

According to a still aspect, the present invention provides a process for preparing polymorph Form F of varenicline tartrate. Polymorph Form F may be prepared by crystallization from a crystallization solvent containing varenicline tartrate. As used herein, the term “crystallization solvent” means a solvent or combination of solvents from which varenicline tartrate is preferentially crystallized as polymorph Form F. Representative crystallization solvents for preparation of Form F include water, acetone and combinations thereof. In a preferred aspect, the crystallization solvent comprises acetone, to which water is gradually added.

In a preferred aspect, Form F of varenicline tartrate may be prepared by slurring starting material, crude or pure varenicline tartrate, anhydrate (e.g., Form B) or solvate, which can be obtained according to the procedures described in U.S. Pat. No. 6,890,927 or U.S. Pat. No. 7,265,119 with acetone or a mixture of acetone and water under heat. The varenicline tartrate is very soluble in water, but not soluble in acetone. The concentration of varenicline tartrate within the solution may range from about 0.1 % by weight to the saturation point. This concentration will, of course, vary depending upon the temperature at which the co-solvent solution is held, with warmer temperatures generally allowing for the preparation of more concentrated solutions of varenicline tartrate. Preferably, the concentration (w/w %) of varenicline tartrate starting material in solution is about 0.5-15%, preferably about 1-10%, more preferably about 1.5-5%. The volume ratio of acetone to water is about 2:0.1-5, preferably about 2:0.2-3, more preferably about 2:0.5-1.5, most preferably about 2:0.8-1.2. Water is then added into the above suspension, and the mixture is heated, suitably to a temperature in the range of from about 45° C. to 85° C., such as about 50° C. to 75° C., for example about 70° C. until all solid materials are dissolved. The clear and hot solution is allowed to cool down to ambient temperature, and the cooled solution is kept at about −20° C.-30° C. for crystallization, preferably at about −10° C.-15° C., and more preferably at about 0-10° C., and most preferably at about 5° C. The crystal Form F of varenicline tartrate is formed over a period of one to six days, and the crystal Form F is recovered from the solvent by filtration. The obtained crystal From F can be dried under a vacuum oven at about 20° C.-60° C., preferably at about 35° C.-50° C., more preferably at about 35° C.-45° C., and most preferably at 38-42° C. for about 10-40 hours to remove the solvent residues.

Once obtained, crystals of polymorph Form F may be used as the nucleating agent or “seed” crystals for subsequent crystallizations of polymorph Form F from the crystallization solvent. In one embodiment, the crystallization solvent is formed by dissolving varenicline tartrate in a hot mixture of acetone and water or other suitable crystallization solvents. The crystallization solvent is then seeded with crystals of polymorph Form F, cooled and filtered, resulting in polymorph Form F. Such seeding with crystals of polymorph Form F may take place at any time during the slurring process. Alternatively, seeding with crystals of polymorph Form F may take place prior to, or simultaneously with, addition of varenicline tartrate to the crystallization solvent.

Form F of crystalline varenicline tartrate as obtained above is characterized by X-ray powder diffraction pattern, substantially the same as shown in FIG. 6 and Table 3.

Form F of varenicline tartrate as obtained above is characterized by differential scanning calorimetry (DSC), substantially the same as shown in FIG. 13.

The crystals of varenicline tartrate obtained from recrystallization in solvents as described in above processes may have different crystal habits (e.g., shape), water contents, surface area, bulk or tap density, or particle size, but they clearly still belonged to a new and novel polymorphic form (Form F) of varenicline tartrate, as it is characterized and confirmed by X-ray powder diffraction pattern, DSC thermogram, TGA and FT-IR spectroscopy techniques. The X-ray powder diffraction pattern of Form E is clearly different from those of other known forms such as Form A, Form B or Form C in the prior art, and also different from those of Form D or Form E in the present application.

According to one aspect, the present invention provides a novel form of varenicline tartrate, e.g., amorphous form of tartrate salt of 5,8,14-triazatetracyclo [10.3.1.0^{2,11}.0^{4,9}]-hexadeca-2(11),3,5,7,9-pentaene (varenicline tartrate or Compound I). The amorphous form of varenicline tartrate herein refers to a solid composition of varenicline tartrate or a drug substance of varenicline tartrate that comprises more than 3% amorphous varenicline tartrate, preferably comprises more than 50% amorphous varenicline tartrate, more preferably contains more than 95% amorphous varenicline tartrate or essentially free of crystalline varenicline tartrate.

More specifically, according to one embodiment, the present invention provides the Compound I drug substance that comprises the amorphous form in a detectable amount. By “drug substance” is meant the active pharmaceutical ingredient (API). The amount of the amorphous form in the drug substance can be quantified by the use of physical methods such as X-ray powder diffraction, solid-state fluorine-19 magic-angle spinning (MAS) nuclear magnetic resonance spectroscopy, solid-state carbon-13 cross-polarization magic-angle spinning (CPMAS) nuclear magnetic resonance spectroscopy, solid state Fourier-transform infrared spectroscopy, and Raman spectroscopy. A detectable amount is an amount that can be detected by such physical methods. The limits of detection of such methods are about 3-5%, and are anticipated to improve with technological advances. The remainder of the drug substance may additionally comprise various crystalline forms of Compound I and polymorphs and pseudopolymorphs thereof. In a class of this embodiment, about 3% to about 100% by weight of the amorphous form is present in the drug substance. In a second class of this embodiment, about 10% to about 100% by weight of the amorphous form is present in the drug substance. In a third class of this embodiment, about 25% to about 100% by weight of the amorphous form is present in the drug substance. In a fourth class of this embodiment, about 50% to about 100% by weight of the amorphous form is present in the drug substance. In a fifth class of this embodiment, about 75% to about 100% by weight of the amorphous form is present in the drug substance. In a sixth class of this embodiment, substantially all of the Compound I drug substance is the amorphous form, i.e., the Compound I drug substance is substantially phase pure amorphous form.

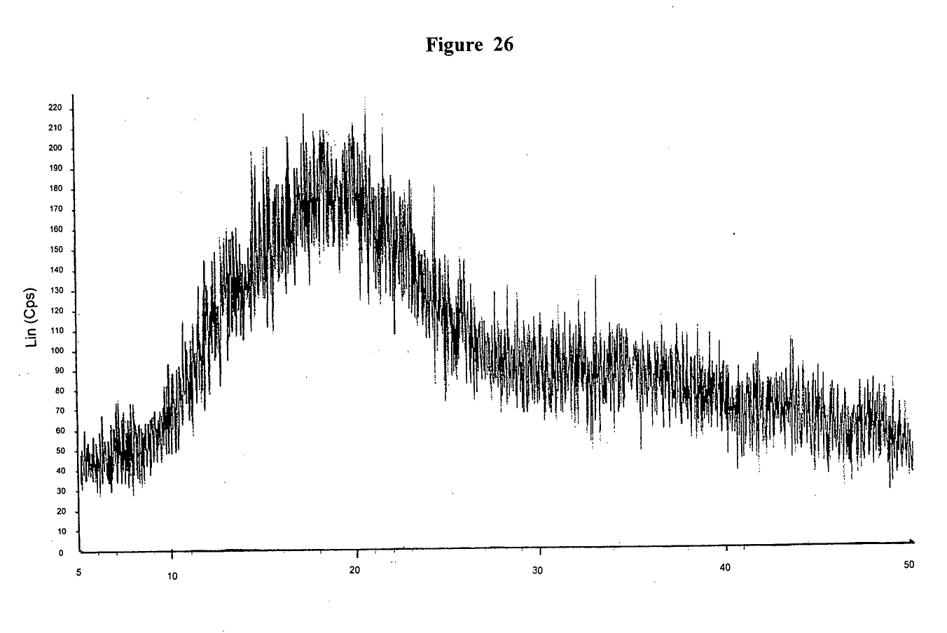

Since the molecule arrangement of amorphous material is in a completely disorder state, the X-ray powder diffraction pattern of amorphous form does not show any discernible or sharp peaks that are characteristics of crystalline material, and only a broad curve is observed, thus demonstrating the amorphous nature of the product. The amorphous form of varenicline tartrate characterized by X-ray powder diffraction pattern is essentially identical to the accompanied drawing of FIG. 26 in the present invention.

The amorphous form of Compound I of the present invention further includes anhydrous amorphous form of Compound I and hydrate amorphous form of Compound I.

According to another aspect, the present invention provides two processes for preparing amorphous form of tartrate salt of 5,8,14-triazatetracyclo[10.3.1.0^{2,11}.0^{4,9}]-hexadeca-2(11),3,5,7,9-pentaene (varenicline tartrate or Compound I).

Accordingly, the first process for preparing amorphous form of varenicline tartrate is a freeze-drying (or lyophilization) method, including the following steps:
a) dissolving crude amorphous or crystalline varenicline tartrate or a mixture of crystalline and amorphous varenicline tartrate in water or a mixture of water and acetone, acetonitrile or in a straight or branched chain C₁-C₄ alcohol solvent,
b) removing the solvent from the solution to obtain solid substance using freeze-drying technique,
c) drying the solid product to obtain amorphous varenicline tartrate.

More specifically, in a first step of the lyophilization process, varenicline tartrate is preferably dissolved in an aqueous (prepared with water) solvent, more preferably dissolved in an aqueous alcohol co-solvent, and most preferably dissolved in water to form a solution.

In particular, varenicline tartrate is highly soluble in water, allowing the complete dissolution of varenicline tartrate at room temperature at a concentration of 50 mg per milliliter (mL). The use of a relatively concentrated solution, e.g. about 50 mg/mL or above 50 mg/mL is therefore preferred.

In a second and preferred step of the lyophilization process, a solution of varenicline tartrate in a solvent is lyophilized to leave a solid residue containing varenicline tartrate in an amorphous state. In this invention, the lyophilization step is performed in two stages: freezing and drying.

In the first stage of lyophilization, the temperature of the solution is decreased until the solution is completely frozen, typically to temperatures as low as minus 50° C., and below, to produce a frozen mixture. Such cooling allows the solute and solvent to separate into separate solid phases. Usually, phase separation will yield a solute in an amorphous state, but may also yield crystalline, microcrystalline or their mixtures. Preferably in this invention, cooling is performed rapidly so that the formation of solute crystals is inhibited, and only amorphous material is formed. More preferably, the solution is cooled using liquid nitrogen with swirling of the vessel containing the solution to coat the wall of the vessel and accelerate freezing. Once the solution has been completely frozen, it is then possible to remove the separated solvent from the frozen mixture by warming up the contents slowly so that the solvent leaves the frozen mixture through sublimation.

The drying stage is preferably conducted under vacuum so that the frozen solvent will vaporize without melting. Heat is applied to transform the frozen solvent into solvent vapor. This vapor migrates through the frozen mixture and escapes into the evacuated space outside of the frozen mixture. The vapor is re-condensed on a refrigerated surface, and turns into a liquid in condenser. The condenser is maintained at a temperature below that of the frozen mixture to drive the drying process.

When the solvent is water, typical lyophilization conditions for producing amorphous form of varenicline tartrate include that the temperature of the frozen mixture is from about −80° C. to about −0° C. before vacuum is applied. The vacuum is typically about 0.05 mm Hg or less, more preferably about 0.01 mm Hg or less and the temperature of the frozen mixture is from about −80° C. to about 20° C. during the drying stage. The drying time using these conditions and standard equipment is dependent on the amount and the nature of solute and solvent used. The drying time is from about 24 hours to about 96 hours for about a 50 g sample of varenicline tartrate dissolved in water.

The obtained product prepared according to the process of the present invention may be characterized by X-ray powder diffraction pattern, as shown in the accompanied drawing of FIG. 26. The X-ray powder diffraction pattern of amorphous form of varenicline tartrate obtained in the present invention does not show any discernible or sharp peaks that are characteristic of crystalline materials; only a flat or broad curve is observed, thus demonstrating the amorphous nature of the product.

The straight or branched chain C₁-C₄ alcohol solvent in the present invention is selected from the group of methanol, ethanol, n-propanol, isopropanol or branched-chain butanols, preferably the alcohol solvent is methanol, ethanol or mixtures thereof. The processes can be carried out with two or more alcohol solvents.

The amorphous form of varenicline tartrate obtained from above process can be anhydrous amorphous varenicline tartrate or hydrate amorphous varenicline tartrate.

In a further aspect, the current invention provides a spray-drying process for preparing the amorphous varenicline tartrate, including following steps: a) dissolving the amorphous or mixture of amorphous and crystalline varenicline tartrate in water, or aqueous alcohol solvent or their mixture of any two solvents thereof; b) stirring the solution until it becomes clear; c) removing the solvent by spray-drying; d) further drying the product under vacuum at elevated temperature until loss of drying is less than 0.5% or constant.

The spray drying process can be carried out using any commercially available dryers, which are used, operates on the principle of nozzle spraying in a parallel flow. For instance, the sprayed product and drying gas flow in the same direction. The drying gas can be air or inert gasses such as nitrogen, argon and carbon dioxide. Nitrogen gas is preferred in this invention. For varenicline tartrate and carrier solution, the spray drying in-let temperature is about 140-180° C., and the out-let temperature is about 90-60° C. at a feed rate of 5-25 ml/min.

Specifically, the amorphous or mixture of amorphous and crystalline varenicline tartrate are usually dissolved in water, aqueous alcohol solvents, such as methanol or ethanol. The concentration of varenicline tartrate is from 1% to 20% (w/v), preferably from 2% to 15%. The weight ratio of active ingredient to carrier is 1:10 to 10:1, preferably 1:2 to 5:1, more preferably 1:1 to 2:1. If necessary, the solution can be heated to completely dissolve the starting materials, and then the solvent is removed by spray drying to obtain the solid product. The solution is cooled to 30° C., and then proceeds with spray drying.

The product obtained from spray drying is further dried to remove the solvent. The product can be dried in a tray drier or dried under vacuum or in a Fluid Bed Dryer. The drying temperature is preferably from 20 to 70° C., drying time is preferably from 8-24 hours. The most preferred drying temperature 35-40° C. and drying time is 12 to 15 hours. After drying, the obtained solid product is the amorphous varenicline tartrate.

We found that amorphous varenicline tartrate can be obtained using this simple and reproducible spray drying process.

According to a process of the invention, the starting material of varenicline tartrate can be obtained by any methods described in the prior art such as U.S. Pat. No. 6,410,500 (or WO 99/35131). The starting material varenicline tartrate can be crude or pure varenicline tartrate, including any solvates or hydrates, preferably purity is more than 95%, more preferably purity is more than 98%, most preferably purity is more than 99%. Starting material varenicline tartrate can be any polymorph forms, including amorphous or crystalline form or their mixture thereof. With the processes where varenicline tartrate goes into solution, the form of the starting material is of minimal relevance since any solid-state structure is lost in solution.

Amorphous varenicline tartrate prepared according to the process of the present invention may be characterized by X-ray powder diffraction pattern, as shown in the accompanied drawing of FIG. 26. The X-ray powder diffraction pattern of amorphous varenicline tartrate does not show any discernible peaks that are characteristic of crystalline materials. The lack of discernible or sharp peaks indicates the characteristic feature of amorphous varenicline tartrate, and also demonstrating the amorphous nature of the obtained product.

The particle sizes of amorphous form of varenicline tartrate of the present invention is about 1-400 μm, preferably about 5-200 μm, more preferably about 10-150 μm, most preferably about 20-100 μm. Small particle sizes are better in improving the blending uniformity or content uniformity of the unit dosage forms such as tablets, particularly in direct compression process.

The amorphous form of varenicline tartrate prepared according to the procedures of the present invention can be used to make pharmaceutical compositions. Therefore, the present invention further provides a pharmaceutical composition for administering effective amount of amorphous form of varenicline tartrate as the active ingredient in unit dosage forms. The unit dosage forms can be administered in a wide variety of oral and parenteral dosage forms, such as by injection, that is, intravenously or intramuscularly. Also, the amorphous form of varenicline tartrate of the present invention can be administered by inhalation, e.g. intranasally or transdermally. It will be obvious to those skilled in the art that the following dosage forms may comprise either amorphous form of varenicline tartrate, as the active component.

More specifically, the present invention also provides pharmaceutical compositions comprising the amorphous Compound I, in association with one or more pharmaceutically acceptable carriers or excipients. In one embodiment the pharmaceutical composition comprises a prophylactically or therapeutically effective amount of the active pharmaceutical ingredient (API) in admixture with pharmaceutically acceptable excipients wherein the API comprises a detectable amount of the amorphous form of the present invention. In a second embodiment the pharmaceutical composition comprises a prophylactically or therapeutically effective amount of the API in admixture with pharmaceutically acceptable excipients wherein the API comprises about 3% to about 100% by weight of amorphous Compound I of the present invention. In a class of this second embodiment, the API in such compositions comprises about 10% to about 100% by weight of amorphous Compound I. In a second class of this embodiment, the API in such compositions comprises about 25% to about 100% by weight of amorphous Compound I. In a third class of this embodiment, the API in such compositions comprises about 50% to about 100% by weight of amorphous Compound I. In a fourth class of this embodiment, the API in such compositions comprises about 75% to about 100% by weight of amorphous Compound I. In a fifth class of this embodiment, substantially all of the API is amorphous Compound I, i.e., the API is substantially phase pure amorphous Compound I. When not comprising substantially phase pure amorphous Compound I, such compositions may additionally comprise various crystalline forms of Compound I and polymorphs and pseudopolymorphs thereof.

The unit dosage forms of pharmaceutical composition comprising amorphous varenicline tartrate or its solid amorphous dispersions can be fast, immediate release or sustained release products, and they can be prepared according to the conventional procedures used in pharmaceutical industry. The details for preparation of tablets are described in Example 10 of the present invention.

According to a further aspect, the present invention provides a pharmaceutical composition, which comprises a prophylactically and a therapeutically effective amount of polymorph Form D of varenicline tartrate, substantially as hereinbefore described, together with one or more pharmaceutically acceptable carriers, diluents or excipients, additives, fillers, lubricants, solvents, binders or stabilizers, optionally, one or more other active ingredients.

According to still another embodiment, the pharmaceutical composition comprises an effective dosage amount of varenicline tartrate, wherein varenicline tartrate comprises at least a certain percentage of polymorph Form D (based on the total amount of varenicline tartrate present in the composition—that is, the total amount of varenicline tartrate being 100%). In other words, at least a certain percentage of varenicline tartrate present within the pharmaceutical composition exists as polymorph Form D, with the remainder of varenicline tartrate being in a different form, including (but not limited to) polymorph Form A, Form B, Form C, Form E or Form F, or any other crystalline, solvate or amorphous form(s). More specifically, trace amounts up to 100% Form D, or any amount in between—for example, the active ingredient or drug substance of varenicline tartrate in the pharmaceutical composition may comprise less than 0.1%, 0.5%, 1%, 2%, 5%, 10%, 20%, 30%, 40% or 50% by weight of Form D based on the total amount of varenicline tartrate in the pharmaceutical composition. Alternatively, the pharmaceutical composition may comprise at least 50%, 60%, 70%, 80%, 90%, 95%, 97%, 98%, 99%, 99.5% or 99.9% by weight of Form D based on the total amount of varenicline tartrate in the pharmaceutical composition.

More specifically, the present invention also provides pharmaceutical compositions comprising the Form D of Compound I, in association with one or more pharmaceutically acceptable carriers or excipients. In one embodiment the pharmaceutical composition comprises a prophylactically or therapeutically effective amount of the active pharmaceutical ingredient (API) in admixture with pharmaceutically acceptable excipients wherein the API comprises a detectable amount of the Form D of the present invention. In a second embodiment the pharmaceutical composition comprises a prophylactically or therapeutically effective amount of the API in admixture with pharmaceutically acceptable excipients wherein the API comprises about 0.1% to about 100% by weight of Form D of Compound I of the present invention. In a class of this second embodiment, the API in such compositions comprises about 5% to about 100% by weight of Form D of Compound I. In a second class of this embodiment, the API in such compositions comprises about 25% to about 100% by weight of Form D of Compound I. In a third class of this embodiment, the API in such compositions comprises about 50% to about 100% by weight of Form D of Compound I. In a fourth class of this embodiment, the API in such compositions comprises about 75% to about 100% by weight of Form D of Compound I. In a fifth class of this embodiment, substantially all of the API is Form D of Compound I, i.e., the API is substantially phase pure Form D of Compound I. When not comprising substantially phase pure Form D of Compound I, such compositions may additionally comprise various crystalline forms of Compound I, other polymorphs and pseudopolymorphs thereof.

According to another aspect, the present invention further provides a pharmaceutical composition, which comprises a prophylactically and a therapeutically effective amount of polymorph Form E of varenicline tartrate, substantially as hereinbefore described, together with one or more pharmaceutically acceptable carriers, diluents or excipients, additives, fillers, lubricants, solvents, binders or stabilizers, optionally, one or more other active ingredients.

According to a still embodiment, the pharmaceutical composition comprises an effective dosage amount of varenicline tartrate, wherein varenicline tartrate comprises at least a certain percentage of polymorph Form E (based on the total amount of varenicline tartrate present in the composition—that is, the total amount of varenicline tartrate being 100%). In other words, at least a certain percentage of varenicline tartrate present within the pharmaceutical composition exists as polymorph Form E, with the remainder of varenicline tartrate being in a different form, including (but not limited to) polymorph Form A, Form B, Form C, Form D or Form F, or any other crystalline, solvate or amorphous form(s). More specifically, trace amounts up to 100% Form E, or any amount in between—for example, the active ingredient or drug substance of varenicline tartrate in the pharmaceutical composition may comprise less than 0.1%, 0.5%, 1%, 2%, 5%, 10%, 20%, 30%, 40% or 50% by weight of Form E based on the total amount of varenicline tartrate in the pharmaceutical composition. Alternatively, the pharmaceutical composition may comprise at least 50%, 60%, 70%, 80%, 90%, 95%, 97%, 98%, 99%, 99.5% or 99.9% by weight of Form E based on the total amount of varenicline tartrate in the pharmaceutical composition.

More specifically, the present invention also provides pharmaceutical compositions comprising the Form E of Compound I, in association with one or more pharmaceutically acceptable carriers or excipients. In one embodiment the pharmaceutical composition comprises a prophylactically or therapeutically effective amount of the active pharmaceutical ingredient (API) in admixture with pharmaceutically acceptable excipients wherein the API comprises a detectable amount of the Form E of the present invention. In a second embodiment the pharmaceutical composition comprises a prophylactically or therapeutically effective amount of the API in admixture with pharmaceutically acceptable excipients wherein the API comprises about 0.1% to about 100% by weight of Form E of Compound I of the present invention. In a class of this second embodiment, the API in such compositions comprises about 5% to about 100% by weight of Form E of Compound I. In a second class of this embodiment, the API in such compositions comprises about 25% to about 100% by weight of Form E of Compound I. In a third class of this embodiment, the API in such compositions comprises about 50% to about 100% by weight of Form E of Compound I. In a fourth class of this embodiment, the API in such compositions comprises about 75% to about 100% by weight of Form E of Compound I. In a fifth class of this embodiment, substantially all of the API is Form E of Compound I, i.e., the API is substantially phase pure Form E of Compound I. When not comprising substantially phase pure Form E of Compound I, such compositions may additionally comprise various crystalline forms of Compound I, other polymorphs and pseudopolymorphs thereof.

According to still another aspect, the present invention further provides a pharmaceutical composition, which comprises a prophylactically and a therapeutically effective amount of polymorph Form F of varenicline tartrate, substantially as hereinbefore described, together with one or more pharmaceutically acceptable carriers, diluents or excipients, additives, fillers, lubricants, solvents, binders or stabilizers, optionally, one or more other active ingredients.

According to another embodiment, the pharmaceutical composition comprises an effective dosage amount of varenicline tartrate, wherein varenicline tartrate comprises at least a certain percentage of polymorph Form F (based on the total amount of varenicline tartrate present in the composition—that is, the total amount of varenicline tartrate being 100%). In other words, at least a certain percentage of varenicline tartrate present within the pharmaceutical composition exists as polymorph Form F, with the remainder of varenicline tartrate being in a different form, including (but not limited to) polymorph Form A, Form B, Form C, Form D or Form E, or any other crystalline, solvate or amorphous form(s). More specifically, trace amounts up to 100% Form F, or any amount in between—for example, the active ingredient or drug substance of varenicline tartrate in the pharmaceutical composition may comprise less than 0.1%, 0.5%, 1%, 2%, 5%, 10%, 20%, 30%, 40% or 50% by weight of Form F based on the total amount of varenicline tartrate in the pharmaceutical composition. Alternatively, the pharmaceutical composition may comprise at least 50%, 60%, 70%, 80%, 90%, 95%, 97%, 98%, 99%, 99.5% or 99.9% by weight of Form F based on the total amount of varenicline tartrate in the pharmaceutical composition.

More specifically, the present invention also provides pharmaceutical compositions comprising the Form F of Compound I, in association with one or more pharmaceutically acceptable carriers or excipients. In one embodiment the pharmaceutical composition comprises a prophylactically or therapeutically effective amount of the active pharmaceutical ingredient (API) in admixture with pharmaceutically acceptable excipients wherein the API comprises a detectable amount of the Form F of the present invention. In a second embodiment the pharmaceutical composition comprises a prophylactically or therapeutically effective amount of the API in admixture with pharmaceutically acceptable excipients wherein the API comprises about 0.1% to about 100% by weight of Form F of Compound I of the present invention. In a class of this second embodiment, the API in such compositions comprises about 5% to about 100% by weight of Form F of Compound I. In a second class of this embodiment, the API in such compositions comprises about 25% to about 100% by weight of Form F of Compound I. In a third class of this embodiment, the API in such compositions comprises about 50% to about 100% by weight of Form F of Compound I. In a fourth class of this embodiment, the API in such compositions comprises about 75% to about 100% by weight of Form F of Compound I. In a fifth class of this embodiment, substantially all of the API is Form F of Compound I, i.e., the API is substantially phase pure Form F of Compound I. When not comprising substantially phase pure Form F of Compound I, such compositions may additionally comprise various crystalline forms of Compound I, other polymorphs and pseudopolymorphs thereof.

In a still aspect, the present invention further provides a solid amorphous dispersion of varenicline tartrate and a carrier or a mixture of one or more carriers. For solid amorphous dispersion of varenicline tartrate and a carrier, the composition comprises more 3% of the amorphous form of varenicline tartrate, preferably more than 50% the amorphous form of varenicline tartrate, most preferably more than 95% or substantially free of any crystalline forms other than its amorphous form.

The solid amorphous dispersion of the present invention has the following characteristics. The amorphous varenicline tartrate is evenly dispersed in the carrier. This highly dispersed material does not contain any crystalline substance, and therefore, it will not induce the crystallization of amorphous material to become a crystalline material. That is, since the amorphous varenicline tartrate is highly dispersed in the amorphous carrier, it will not convert back into crystalline varenicline tartrate. The solid amorphous dispersion of varenicline tartrate is also stable, and has a good material flow property and high bulk & tap density, and thus it is particularly suitable for preparation of pharmaceutical composition.

The carrier used to make the solid amorphous dispersion of the present invention may be an amorphous material or it can be converted into amorphous material. The suitable carriers should be soluble in water, methanol, ethanol, acetonitrile, acetone and dichloromethane or mixtures thereof. The suitable carriers should be pharmaceutically acceptable as well. The carriers of the present invention include various types of polyethylene glycols (PEG), polyvinylpyrrlidones (PVP) and its derivatives such as cross-linked PVP or PVP-VA64, sugars, lactose, starches, mannitol, methylcellulose, hydroxylethylcellulose, hydroxylmethylcellulose, ethylcellulose, hydroxylpropylmethylcellulose (HPMC) or other cellulose derivatives. The preferred polyethylene glycols (PEG) are PEG 4000, PEG 5000, PEG 6000 or PEG 8000. The preferred polyvinylpyrrolidones (PVP) are PVP K15 or PVP K30.

Additional suitable carriers to make the solid amorphous dispersion of the present invention include α-cyclodextrin, β-cyclodextrin, hydroxylpropyl-β-cyclodextrin, preferably hydroxylpropyl-β-cyclodextrin. The suitable carriers to make the solid amorphous dispersion of the present invention should be commercially available or can be made by known procedures.

According to another aspect, the present invention provides a process for preparing a solid amorphous dispersion of varenicline tartrate and a carrier using distillation or spray drying technique, and the details can be found in examples of the present inventions. Distillation technique for preparing solid amorphous dispersion includes the following steps: dissolving the starting materials of varenicline tartrate in a solvent, e.g. water, methanol, ethanol, acetone, dichloromethane or mixtures thereof at heating, preferably heated to boiling point of the solvent; evaporating the solvent under the reduced pressure to dryness; grinding the solid residues; and drying the product under vacuum at 35-45° C. Spray drying technique for preparing solid amorphous dispersion comprises the following steps: dissolving the starting material of varenicline tartrate in a solvent, e.g. water, methanol, ethanol, acetone, dichloromethane or mixtures of solvents thereof at heating; cooling the solution to 30° C.; removing the solvent by spray drying to afford solid residues; drying the product under vacuum at 35-45° C. Alternatively, the varenicline tartrate and carrier solution can be prepared by separately dissolving varenicline tartrate or carrier in a solvent to give individual solution, and then varenicline tartrate solution can be added into the carrier solution or vice versa to afford a solution of varenicline tartrate and carrier.

The solid amorphous dispersion of varenicline tartrate and a carrier and pharmaceutically acceptable excipients can be used to prepare pharmaceutical compositions. Therefore, according to a further aspect, the present invention provides a pharmaceutical composition for administering effective amount of amorphous varenicline tartrate in a form of solid amorphous dispersion in unit dosage forms. The unit dosage forms can be administered in a wide variety of oral and parenteral dosage forms as described above. Additionally, it will be obvious to those skilled in the art that the following dosage forms may comprise either amorphous varenicline tartrate or a corresponding pharmaceutically acceptable salt of a compound of the present invention as the active component.

Pharmaceutical compositions as provided by the present invention can be prepared by known procedures using well-known and readily available ingredients. In preparation of compositions as provided by the present invention, amorphous form, polymorph Form D, Form E or Form F of crystalline varenicline tartrate, substantially as hereinbefore described, can be mixed with one or more carriers, excipients, diluents, additives, fillers, lubricants, solvents, binders or stabilizers, optionally, one or more other active ingredients.

Pharmaceutical compositions as provided by the present invention can be in the form of tablets, pills, powders, lozenges, sachets, cachets, elixirs, suspensions, emulsions, solutions, syrups, aerosol, ointments soft and hard gelatin capsules, suppositories, sterile injectable solutions and sterile packaged powders containing, for example, up to 50% by weight of amorphous form, polymorph Form D, Form E or Form F, substantially as hereinbefore described.

Some examples of suitable carriers, excipients, and diluents include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water syrup, methyl cellulose, methyl- and propylhydroxybenzoates, talc, magnesium stearate and mineral oil. The compositions can additionally include lubricating agents, wetting agents, and emulsifying and suspending agents, preserving agents, sweetening agents or flavoring agents.

The pharmaceutical compositions of the invention may be formulated so as to provide quick, extended, sustained or delayed release of amorphous form, polymorph Form D, Form E or Form F of varenicline tartrate, substantially as hereinbefore described, after administration to the patient by employing procedures well known in the art. The pharmaceutical compositions of the invention may be preferably formulated so as to provide quick (or immediate), delayed, extended or sustained release tablets consisting of amorphous form, polymorph Form D, Form E or Form F of varenicline tartrate, substantially as hereinbefore described as active ingredient and plus any additional excipients suitable for preparation of quick, delayed, extended or sustained release tablets.

According to one preferred aspect, the pharmaceutical composition is a quick release formulation. For example, a quick release formulation may comprise lactose or dicalcium phosphate as main diluents, amorphous form, crystalline polymorph Form D, Form E or Form F of varenicline tartrate as active ingredient, microcrystalline cellulose as a binder or filler, a disintegratant and a lubricant. The dose units are preferably coated with a film coating.

According to one preferred aspect, the pharmaceutical composition is an extended release formulation. For example, an extended release formulation may comprise spheroids comprised of amorphous form, crystalline polymorph Form D, Form E or Form F of varenicline tartrate, microcrystalline cellulose, and, optionally, hydroxypropylmethylcellulose. The spheroids are preferably coated with a film coating composition comprised of ethyl cellulose and hydroxypropylmethylcellulose.

According to another preferred embodiment, the pharmaceutical composition is a sustained release formulation (e.g., in the form of a tablet). The sustained release formulation may comprise amorphous form, crystalline polymorph Form D, Form E or Form F of varenicline tartrate, a release rate controlling excipient, and optionally other adjuvants. Suitable rate controlling excipients include, but are not limited to, hydroxyalkyl cellulose, such as hydroxypropyl cellulose and hydroxypropyl methyl cellulose (HPMC); poly(ethylene) oxide; alkylcellulose, such as ethyl cellulose and methyl cellulose; carboxymethyl cellulose; hydrophilic cellulose derivatives; carboxyvinylpolymers (e.g., Carbopol 971P), polyvinylpyrrolidone (PVP) derivatives and polyethylene glycol derivatives.

According to still another preferred embodiment, the pharmaceutical composition is a sustained release formulation, e.g., osmotic drug delivery system using asymmetric membrane technology (AMT). The osmotic drug delivery formulation may comprise amorphous form, crystalline polymorph Form D, Form E or Form F of varenicline tartrate, a release rate controlling excipient, e.g., pore-form materials, and optionally other adjuvants. In asymmetric membrane system, a water-insoluble polymer is combined with a water-soluble, pore-forming material, to form porous coatings. The pore-former materials can be any suitable inorganic materials and polymeric materials.

The sustained release pharmaceutical composition comprises about 0.01-50 mg of amorphous form, polymorphs Form D, Form E or Form F of varenicline tartrate and about 5% w/w to about 70% w/w of a release rate controlling pharmaceutical excipients. A preferred sustained release pharmaceutical composition comprises from about 0.5-3 mg of amorphous form, crystalline polymorphs Form D, Form E or Form F of varenicline tartrate and about 10 w/w to about 66% w/w of water-insoluble polymer comprising a cellulose derivatives such as hydroxyprpopyl methylcellulose, methyl cellulose or ethyl cellulose, along with water-soluble cellulose derivatives such as acacia, dextrin, PEGs, starch polyacrylates or polyvinyl alcohols.

The pharmaceutical compositions are preferably formulated in a unit dosage form, each dosage containing from about 0.01 to about 50 mg, more usually about 0.5 to about 3 mg, of amorphous form, polymorph Form D, Form E or Form F of varenicline tartrate, substantially as hereinbefore described. The term “unit dosage form” refers to physically discrete units suitable as unitary dosages for human subjects and other mammals, each unit containing a predetermined quantity of active material calculated to produce the desired therapeutic effect, in association with a suitable pharmaceutical carrier.

In powders, the excipient is a finely divided solid that is mixed with the finely divided active component. In tablets, the active component is mixed with the carrier having necessary binding properties in suitable proportions and compacted in the shape and size desired.

Suitable carriers are magnesium carbonate, magnesium stearate, talc, sugar or lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component, with or without other carriers, is surrounded by a carrier, which is thus in association with it. Similarly, tablets, powders, capsules, pills, cachets, and lozenges can be used as solid dosage forms suitable for oral administration.

Tablets can be made by compressing active ingredient powders or particles and a binder mixed with one or more excipients through a tablet compression machine. The suitable excipients are sodium hydroxylmethylcelluse, lubricants, inert excipients, preservatives and disintegrating agents (e.g., sodium starch glycolate, cross-polyvinylpyrrolidone, sodium methylcelluse carboxlate) or dispersion. Tablets can also be coated with suitable coating agents, and they may be immediate release or sustained release drug products.

For preparing suppositories, a low melting wax, such as a mixture of fatty acid glycerides or cocoa butter, is first melted and the active component is then dispersed homogeneously therein, as by stirring. The homogenous mixture is then poured into molds with suitable size, allowed to cool, and thereby to solidify.

The pharmaceutical composition of the present invention is preferably in unit dosage form. In such form, the preparation is subdivided into unit doses containing appropriate quantities of the active component. The unit dosage form can be a packaged preparation, and the package contains discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

A further aspect of the present invention relates to a method of treating or preventing patients suffering from inflammatory bowel disease (including but not limited to ulcerative colitis, pyoderma gangrenosum and Crohn's disease), irritable bowel syndrome, spastic dystonia, chronic pain, acute pain, celiac sprue, pouchitis, vasoconstriction, anxiety, panic disorder, depression, bipolar disorder, autism, sleep disorders, jet lag, amyotrophic lateral sclerosis (ALS), cognitive dysfunction, hypertension, bulimia, anorexia, obesity, cardiac arrhythmias, gastric acid hypersecretion, ulcers, pheochromocytoma, progressive supranuclear palsy, chemical dependencies and addictions (e.g., dependencies on, or addictions to nicotine (and/or tobacco products), alcohol, benzodiazepines, barbiturates, opioids or cocaine), headache, migraine, stroke, traumatic brain injury (TBI), obsessive-compulsive disorder (OCD), psychosis, Huntington's chorea, tardive dyskinesia, hyperkinesia, dyslexia, schizophrenia, multi-infarct dementia, age-related cognitive decline, epilepsy, including petit mal absence epilepsy, senile dementia of the Alzheimer's type (AD), Parkinson's disease (PD), attention deficit hyperactivity disorder (ADHD) and Tourette's Syndrome, particularly, nicotine dependency, addition and withdrawal; including use in smoking cessation therapy, and certain complications thereof, comprising administering to a patient in need of such treatment an effective amount of a pharmaceutical composition comprising amorphous form, polymorph Form D, Form E or Form F of varenicline tartrate and a pharmaceutically acceptable carrier.

The present invention further provides amorphous form, polymorph Form D, Form E or Form F of varenicline tartrate, for use in the manufacture of a medicament for the treatment and/or prophylaxis of patients suffering from inflammatory bowel disease (including but not limited to ulcerative colitis, pyoderma gangrenosum and Crohn's disease), irritable bowel syndrome, spastic dystonia, chronic pain, acute pain, celiac sprue, pouchitis, vasoconstriction, anxiety, panic disorder, depression, bipolar disorder, autism, sleep disorders, jet lag, amyotrophic lateral sclerosis (ALS), cognitive dysfunction, hypertension, bulimia, anorexia, obesity, cardiac arrhythmias, gastric acid hypersecretion, ulcers, pheochromocytoma, progressive supranuclear palsy, chemical dependencies and addictions (e.g., dependencies on, or addictions to nicotine (and/or tobacco products), alcohol, benzodiazepines, barbiturates, opioids or cocaine), headache, migraine, stroke, traumatic brain injury (TBI), obsessive-compulsive disorder (OCD), psychosis, Huntington's chorea, tardive dyskinesia, hyperkinesia, dyslexia, schizophrenia, multi-infarct dementia, age-related cognitive decline, epilepsy, including petit mal absence epilepsy, senile dementia of the Alzheimer's type (AD), Parkinson's disease (PD), attention deficit hyperactivity disorder (ADHD) and Tourette's Syndrome, particularly, nicotine dependency, addition and withdrawal; including use in smoking cessation therapy, and certain complications thereof.

The particular dose of amorphous form, polymorph Form D, Form E or Form F of varenicline tartrate, substantially as hereinbefore described, administered according to this invention will of course be determined by the particular circumstances surrounding the case, the route of administration, the particular condition being treated, and similar considerations.

Though the present invention has described details of various embodiments, it will be obvious to those skilled in the art that similar situations, modifications or amendments should be included in the present invention as well. The following examples are provided to illustrate specific embodiments of the present invention, they are not intended to be limiting in any way.

### Experimental

Thermogravimetric analysis (TGA) measurements were performed in a Pyris I TGA of Perkin-Elmer (TGA7) under nitrogen purge. The sample was heated from 20° C. to 200° C. at a scan rate of 10° C./minute.

DSC measurements were performed in a TA instrument with a sealed pan at a scan rate of 10° C./minute from 40° C. to 260° C. under nitrogen purge.

X-ray powder diffraction (X-RPD) data were obtained by ARL X-Ray powder diffractometer model XTRA-030. Scanning range 3-50 deg. 2 theta, continuous scan rate 3 deg./min. The accuracy of peak positions was defined as ±0.2 degrees due to such experimental differences as instrumentation and sample preparation etc.

Fourier transform infrared (FT-IR) spectrum was recorded on Thermo Nicolet Corp.'s NEXUS (470 FT-IR) under nitrogen purge using KBr as carrier or medium. Total scan was 32 and resolution is 4.

### EXAMPLES

### Example 1

### Preparation of Form B of Varenicline Tartrate

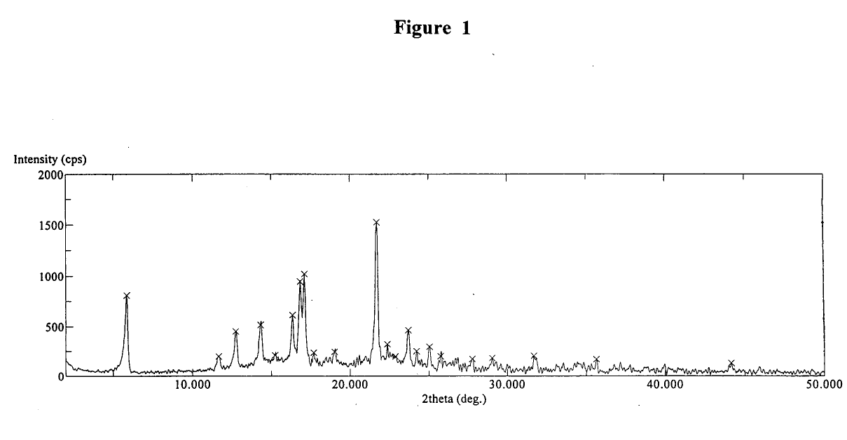

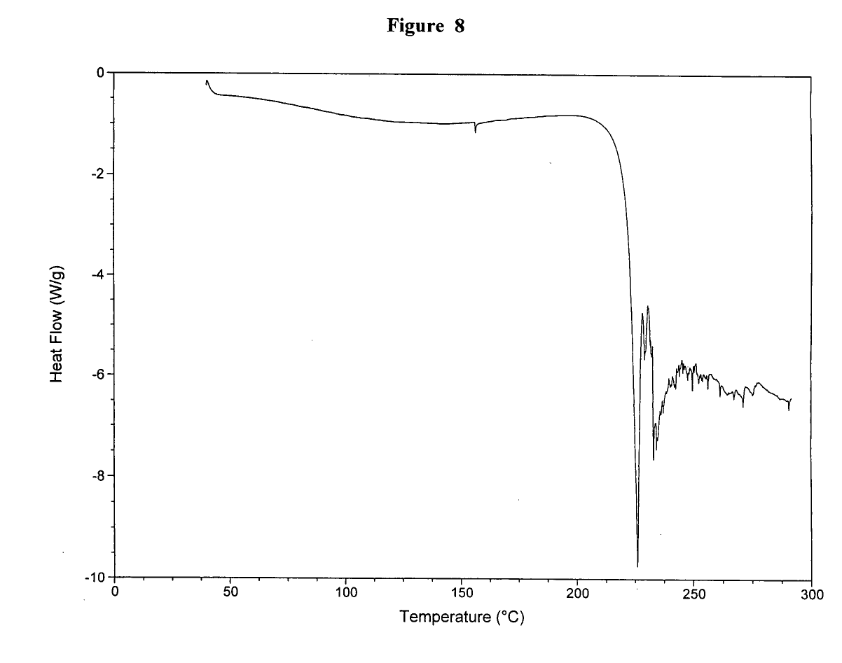

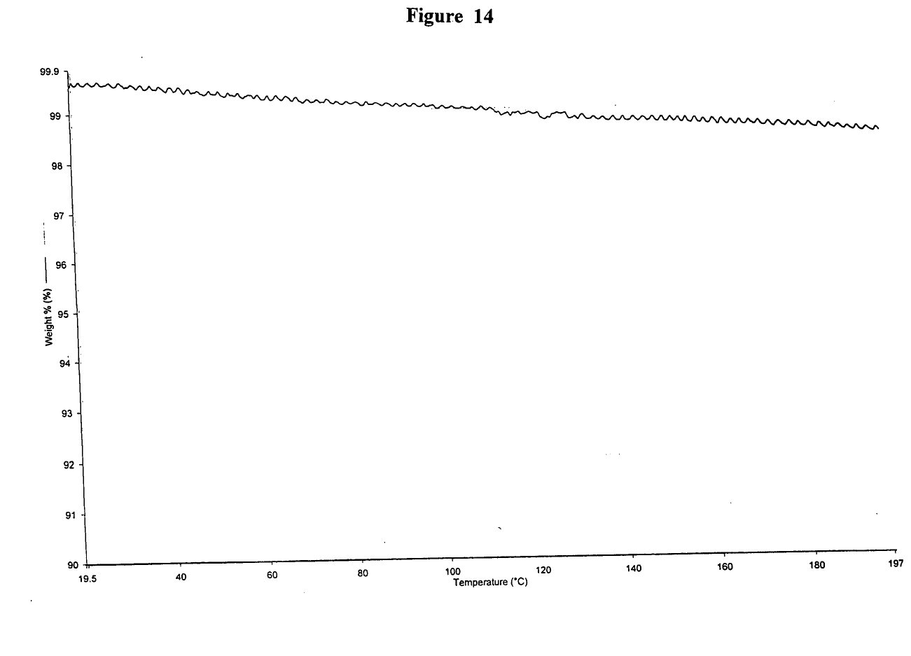

Form B of varenicline tartrate (20.0 g) was obtained by following a procedure described in U.S. Pat. No. 7,265,119 (Example 1, column 29, line 5-40). DSC, FT-IR, TGA and X-ray diffraction pattern techniques were used to characterize the obtained product. DSC experiment of the obtained product showed an endothermic peak at about 225.91° C., as shown in FIG. 8. Powder X-ray diffraction pattern of the obtained product is shown in FIG. 1, essentially same as that of Form B described in U.S. Pat. No. 7,265,119. The TGA, as shown in FIG. 14, indicated that the obtained product contains less than about 0.5% w/w water residue at 60-160° C. The FT-IR spectrum is shown in FIG. 20. Therefore, the obtained product is confirmed as a known polymorph of anhydrous varenicline tartrate (Form B).

### Example 2

### Preparation of Form C of Varenicline Tartrate

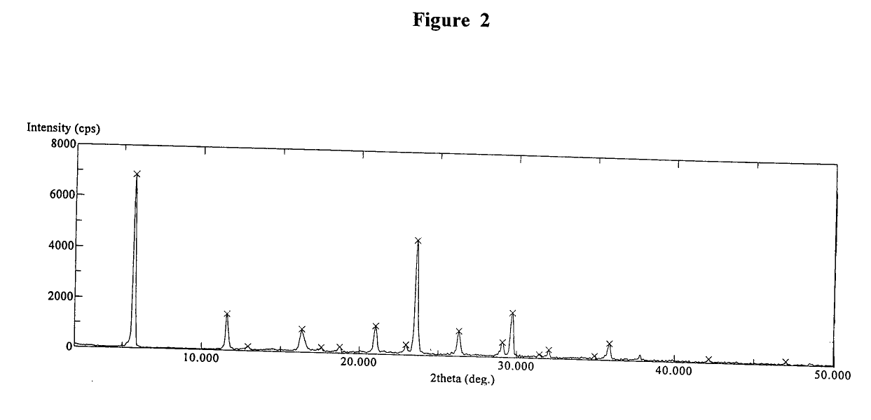

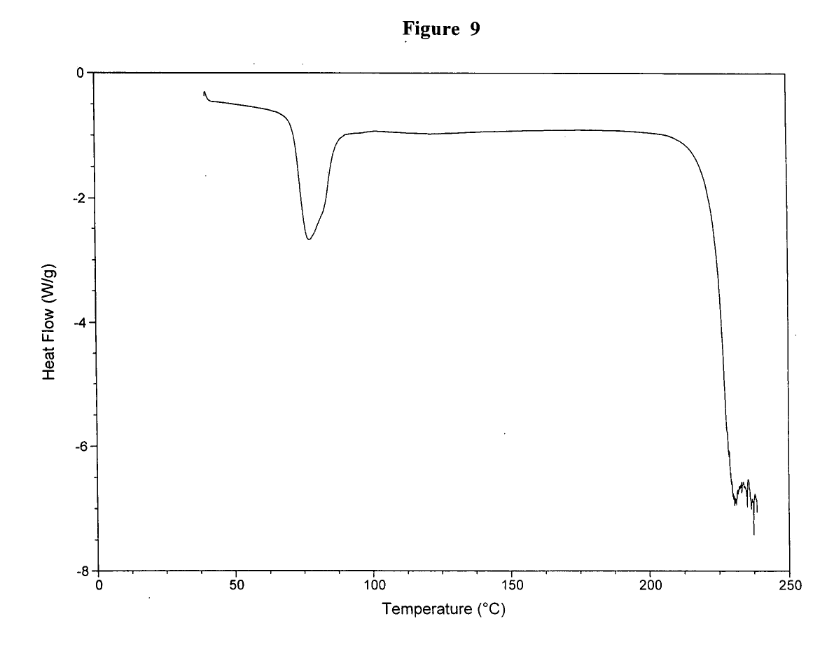

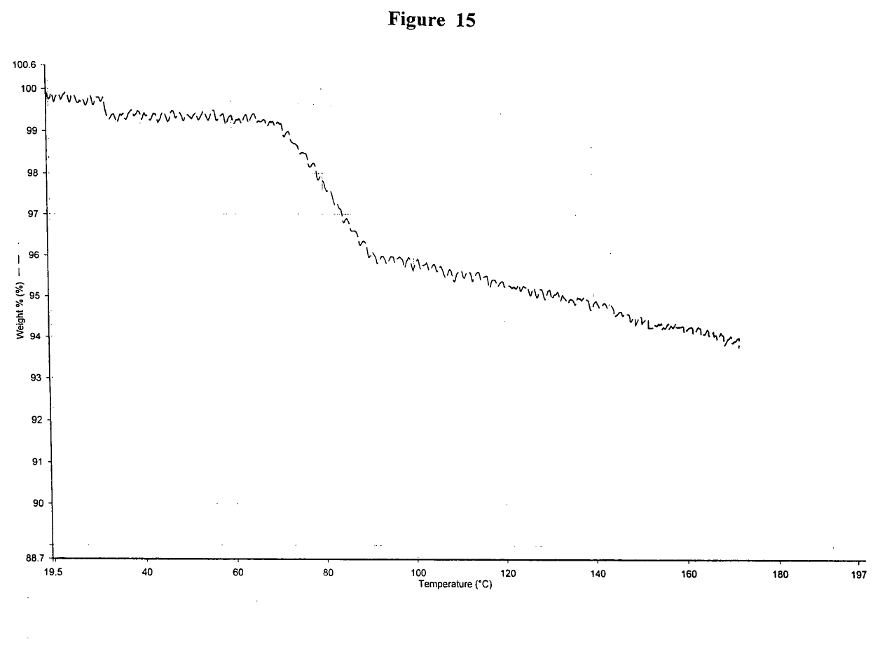

Varenicline tartrate (3.0 g) was suspended in about 20 ml boiling acetonitrile (HPLC grade). To the suspension was added about 10 ml water and the suspension was heated up until all solid materials are dissolved. The resulting clear solution was then cooled down to ambient temperature and kept at 5° C. for recrystallization for overnight and some crystals were formed. The recrystallization continued at about 5° C. for four additional days. The resulting crystals were isolated by filtration and dried in vacuum oven at about 40° C. for 6 hours, and then kept at ambient temperature for two days, and subsequently dried under vacuum oven at about 40° C. for 7 hours and then at 25° C. for overnight to give a white crystalline solid (about 2.5 g). DSC, FT-IR, TGA and X-ray diffraction pattern techniques were used to characterize the obtained product. DSC experiment of the obtained product showed an endothermic peak at about 77.23° C. and another endothermic peak at about 225° C., as shown in FIG. 9. Powder X-ray diffraction pattern of the obtained product is shown in FIG. 2, essentially same as that of Form C described in U.S. Pat. No. 7,265,119. The TGA, as shown in FIG. 15, indicated that the obtained product contains about 5% w/w water. The FT-IR spectrum is shown in FIG. 21. Therefore, the obtained product is confirmed as a known polymorph of varenicline tartrate monohydrate (Form C).

### Example 3

### Preparation of Form D of Varenicline Tartrate

Varenicline tartrate (2.0 g) was suspended in about 20 ml boiling methanol (HPLC grade). To the suspension was added about 6 ml water and the suspension was heated up until all solid materials are dissolved. The resulting clear solution was then cooled down to ambient temperature and kept at 5° C. for recrystallization for overnight and some crystals were formed. The recrystallization continued at about 5° C. for four additional days. The resulting crystals were isolated by filtration and dried in vacuum oven at about 40° C. for 6 hours, and then kept at ambient temperature for two days, and subsequently dried under vacuum oven at about 40° C. for 7 hours and then at 25° C. for overnight to give a white crystalline solid (about 1.5 g). DSC, FT-IR, TGA and X-ray diffraction pattern techniques were used to characterize the obtained product. DSC experiment of the obtained product showed an endothermic peak at about 225° C., as shown in FIG. 10. Powder X-ray diffraction pattern of the obtained product is shown in FIG. 3. The TGA, as shown in FIG. 16, indicated that the obtained product contains less than about 0.3% w/w water residue from 60-160° C. The FT-IR spectrum is shown in FIG. 22. Therefore, the obtained product is confirmed as a new polymorph of anhydrous varenicline tartrate (Form D).

### Example 4

### Preparation of Form D of Varenicline Tartrate

Varenicline tartrate (2.0 g) was suspended in about 20 ml boiling N,N-dimethylformamide (HPLC grade). To the suspension was added about 8 ml water and the suspension was heated up until all solid materials are dissolved. The resulting clear solution was then cooled down to ambient temperature and kept at 5° C. for recrystallization for overnight and some crystals were formed. The recrystallization continued at about 5° C. for four additional days. The resulting crystals were isolated by filtration and dried in vacuum oven at about 40° C. for 6 hours, and then kept at ambient temperature for two days, and subsequently dried under vacuum oven at about 40° C. for 7 hours and then at 25° C. for overnight to give a white crystalline solid (about 1.5 g). DSC, FT-IR, TGA and X-ray diffraction pattern techniques were used to characterize the obtained product. DSC experiment of the obtained product showed an endothermic peak at about 225.76° C., as shown in FIG. 11. Powder X-ray diffraction pattern of the obtained product is shown in FIG. 4. The TGA, as shown in FIG. 17, indicated that the obtained product contains less than about 0.8% w/w water or solvent residue from 60-160° C. The FT-IR spectrum is shown in FIG. 23. Therefore, the obtained product is confirmed as a new polymorph of anhydrous varenicline tartrate (Form D).

### Example 5

### Preparation of Form E of Varenicline Tartrate

Varenicline tartrate (2.0 g) was suspended in about 25 ml isopropanol (IPA) (HPLC grade). To the suspension was added about 5 ml water and the suspension was heated up until all solid materials are dissolved. The resulting clear solution was then cooled down to ambient temperature and kept at 5° C. for recrystallization for overnight and some crystals were formed. The recrystallization continued at about 5° C. for four additional days. The resulting crystals were isolated by filtration and dried in vacuum oven at about 40° C. for 6 hours, and then kept at ambient temperature for two days, and subsequently dried under vacuum oven at about 40° C. for 7 hours and then at 25° C. for overnight to give a white crystalline solid (about 1.0 g). DSC, FT-IR, TGA and X-ray diffraction pattern techniques were used to characterize the obtained product. DSC experiment of the obtained product showed an endothermic peak at 85.17° C. and another endothermic peak at about 225.82° C., as shown in FIG. 12. Powder X-ray diffraction pattern of the obtained product is shown in FIG. 5. The TGA, as shown in FIG. 18, indicated that the obtained product contains about 5.0% w/w water from 60-160° C. The FT-IR spectrum is shown in FIG. 24. Therefore, the obtained product is confirmed as a new polymorph of varenicline tartrate monohydrate (Form E).

### Example 6

### Preparation of Form F of Varenicline Tartrate

Varenicline tartrate (2.0 g) was suspended in about 20 ml acetone (HPLC grade). To the suspension was added about 10 ml water and the suspension was heated up until all solid materials are dissolved. The resulting clear solution was then cooled down to ambient temperature and kept at 5° C. for recrystallization for overnight and some crystals were formed. The recrystallization continued at about 5° C. for four additional days. The resulting crystals were isolated by filtration and dried in vacuum oven at about 40° C. for 6 hours, and then kept at ambient temperature for two days, and subsequently dried under vacuum oven at about 40° C. for 7 hours and then at 25° C. for overnight to give a white crystalline solid (about 1.1 g). DSC, FT-IR, TGA and X-ray diffraction pattern techniques were used to characterize the obtained product. DSC experiment of the obtained product showed an endothermic peak at 85.37° C. and another endothermic peak at about 225° C., as shown in FIG. 13. Powder X-ray diffraction pattern of the obtained product is shown in FIG. 6. The TGA, as shown in FIG. 19, indicated that the obtained product contains about 5.0% w/w water from 60-160° C. The FT-IR spectrum is shown in FIG. 25. Therefore, the obtained product is confirmed as a new polymorph of varenicline tartrate monohydrate (Form F).

### Example 7

### Preparation of Form C of Varenicline Tartrate

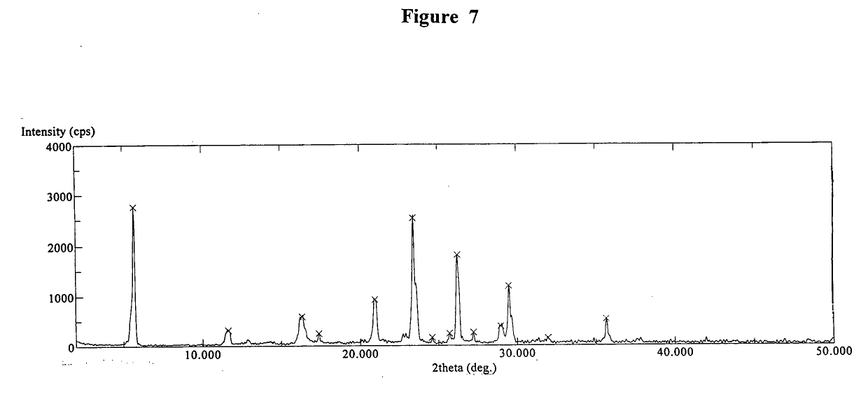

Varenicline tartrate (2.0 g) was suspended in about 20 ml ethanol (HPLC grade). To the suspension was added about 10 ml water and the suspension was heated up until all solid materials are dissolved. The resulting clear solution was then cooled down to ambient temperature and kept at 5° C. for recrystallization for overnight and some crystals were formed. The recrystallization continued at about 5° C. for four additional days. The resulting crystals were isolated by filtration and dried in vacuum oven at about 40° C. for 6 hours, and then kept at ambient temperature for two days, and subsequently dried under vacuum oven at about 40° C. for 7 hours and then at 25° C. for overnight to give a white crystalline solid (about 1.3 g). DSC and X-ray diffraction pattern techniques were used to characterize the obtained product. DSC experiment of the obtained product showed an endothermic peak at 83.25° C. and another endothermic peak at about 225° C. Powder X-ray diffraction pattern of the obtained product is shown in FIG. 7. Therefore, the obtained product is confirmed as a known polymorph of varenicline tartrate monohydrate (Form C).

### Example 8

### Preparation of Amorphous Varenicline Tartrate

Method A: Varenicline tartrate (5 g) was completely dissolved in water (50 ml) in a round bottom flask to obtain a clear solution. The solution was then transferred to a heavy walled lyophilization flask (2 liters). The solution in flask is rapidly cooled by liquid nitrogen until it is frozen. The lyophilizer was evacuated and maintained under about 0.01 mm Hg vacuum for about 9-10 hours. The sample was submitted for powder X-ray analysis, which confirmed that the resulting substance was in amorphous form.

Method B: Varenicline tartrate (5.0 g) was dissolved in 60 ml of water, and the suspension mixture is heated to 40-50° C. to obtain a clear solution. The hot solution was cooled to ambient temperature (25-30° C.), and then subjected to spray drying in a Mini-Spray Dryer (e.g., Buchi Model-190) at an inlet temperature 118-145° C. and outlet temperature 72-87° C. using nitrogen gas. A light-white powder of varenicline tartrate in an amorphous form was obtained. The product was further dried under vacuum at 30-45° C. for 10 hours to afford 4.5 g of the desired product of amorphous varenicline tartrate. The powder X-ray diffractogram showed that the resulting substance was in amorphous form.

### Example 9

### Preparation of Solid Amorphous Dispersion of Varenicline Tartrate and PVP

Method A: Varenicline tartrate (3.0 g) and polyvinylpyrrolidone (PVP, K=30) (6.0 g) was dissolved in 200 ml of water and ethanol (1:1, v/v), and the suspension mixture is heated to 40-50° C. to obtain a clear solution. The hot solution was cooled to ambient temperature (25-30° C.), and then subjected to spray drying in a Mini-Spray Dryer (e.g., Buchi Model-190) at an inlet temperature 119-148° C. and outlet temperature 75-85° C. using nitrogen gas. A light-white powder of solid dispersion containing varenicline tartrate and PVP in an amorphous form was obtained. The product was further dried under vacuum at 40° C. for 24 hours to afford 8.0 g of the desired solid amorphous dispersion of varenicline tartrate and PVP. The powder X-ray diffractogram showed that the resulting substance was in amorphous form.

Method B: 3.0 g varenicline tartrate and 6.0 g PVP-VA64 (Plastone S-630, K=26-34) was dissolved in ethanol (150 ml) at ambient temperature, and suspension mixture was heated to 50° C. to obtain a clear solution. The solvent was evaporated through distillation under vacuum (30-80 mm Hg) at about 40° C. to about 70° C. The product was then isolated (about 8.5 g) when no visible liquid was remained and drying was continued under vacuum at about 40° C. for 24-48 hours to remove the solvent. The powder X-ray diffractogram and DSC of the solid material showed that the resulting substance was amorphous product.

### Example 10

### Preparation of Pharmaceutical Composition Comprising Amorphous Varenicline Tartrate

Pharmaceutical compositions for tablet dosage forms comprising amorphous varenicline tartrate or its solid amorphous dispersion and excipients are described in Table 4.

TABLE 4
Formulations A, B and C of 1 mg Varenicline Tartrate Tablets

| | mg/per tablet | | |
|---|---|---|---|
| Ingredients | A | B | C |
| Core: | | | |
| Amorphous varenicline tartrate | 1 | — | 3 |
| Dispersion of varenicline tartrate and PVP (K30) | — | 3 | — |
| Mirocrystalline cellulose | 50 | 30 | 30 |
| Calcium phosphate (A-Tab) | 50 | 60 | 50 |
| Sodium starch glycolate | 15 | — | — |
| Pregelatinized starch | — | 24 | 32 |
| Magnesium stearate | 3 | 3 | 3 |
| Coating | | | |
| Coating agent (HPMC)(sustained release) | — | — | 12 |
| Coating agent (opadry white), water* | 6 | 6 | — |
| (Immediate release) | | | |
| Total tablet weight (mg) | 125 | 125 | 130 |

*Evaporated during process

Table 4 lists three formulations of tablets: formulation A and B are for immediate release product, and formulation C is for sustained release product. Formulation B is for tablets comprising solid amorphous dispersion of varenicline tartrate and PVP. There are two major steps involved in manufacturing tablets (1.0 mg active ingredient): (1) preparation of varenicline tartrate tablet core; (2) coating the tablet core.

For making tablets, the amorphous varenicline tartrate or the solid amorphous dispersion of varenicline tartrate and carrier are sifted by a clean screen (typically 0.066″), and other excipients are sifted by another screen (typically 0.080″). The sieved materials are then mixed in a tumbler at a speed of 100 rpm for 15 minutes, to afford completely homogeneous materials. The mixed materials are compressed into tablet cores using tablet-manufacturing equipments. The tablet cores are then placed on the tablet-coating machine, which is pre-heated to 60° C. for coating. Prior to spraying the coating agents, the coating pan's speed is adjusted to 5-9 rpm and the exhaustive temperature is maintained at 40-50° C. The coated tablets are dried for 5-10 minutes. The tablets are placed in the induction-sealed bottles with desiccants.

We claim:

1. The polymorph Form D of crystalline varenicline tartrate.

2. The polymorph Form D of claim 1, characterized substantially as having an X-ray diffraction pattern with characteristic peaks (expressed in 2θ±0.2° 2θ) at one or more of the following positions: 5.66, 11.48, 14.12, 16.10, 16.90, 17.46, 20.90, 21.50, 23.42, 29.04, 29.42 or 31.60.

3. The polymorph Form D of claim 1, characterized as having X-ray powder diffraction pattern substantially the same as that shown in FIG. 3 or FIG. 4.

4. The polymorph Form D of claim 1, characterized as having an endothermic peak at about 224-228° C. in differential scanning calorimetry (DSC) and being substantially the same as that shown in FIG. 10 or FIG. 11.

5. The polymorph Form D of claim 1, characterized as having transform infrared (FT-IR) spectrum substantially the same as that shown in FIG. 22 or FIG. 23.

6. The polymorph Form E of crystalline varenicline tartrate.

7. The polymorph Form E of claim 6, characterized substantially as having an X-ray diffraction pattern with characteristic peaks (expressed in 2θ±0.2° 2θ) at one or more of the following positions: 5.44, 5.88, 11.62, 16.48, 21.16, 23.54, 23.82, 24.84, 26.30, 29.30, 29.62, 29.90, 35.80 or 36.08.

8. The polymorph Form E of claim 6, characterized as having X-ray powder diffraction pattern substantially the same as that shown in FIG. 5.

9. The polymorph Form E according to claim 6, characterized as having an endothermic peak at about 82-88° C. and another endothermic peak at about 224-228° C. in differential scanning calorimetry (DSC) and being substantially the same as that shown in FIG. 12.

10. The polymorph Form E of claim 6, characterized as having transform infrared (FT-IR) spectrum substantially the same as that shown in FIG. 24.

11. The polymorph Form E of claim 6, wherein the crystalline form is hydrate.

12. The polymorph Form E of claim 11, wherein the hydrate is a monohydrate.

13. The polymorph Form F of crystalline varenicline tartrate.

14. The polymorph Form F of claim 13, characterized substantially as having an X-ray diffraction pattern with characteristic peaks (expressed in 2θ±0.2° 2θ) at one or more of the following positions: 5.68, 11.60, 16.32, 21.08, 23.38, 23.60, 26.34, 29.62 or 31.94.

15. The polymorph Form F of claim 13, characterized as having X-ray powder diffraction pattern substantially the same as that shown in FIG. 6.

16. The polymorph Form F of claim 13, characterized as having an endothermic peak at about 82-88° C. and another endothermic peak at about 224-228° C. in differential scanning calorimetry (DSC) and being substantially the same as that shown in FIG. 13.

17. The polymorph Form F according to claim 13, characterized as having transform infrared (FT-IR) spectrum substantially the same as that shown in FIG. 25.

18. The polymorph Form F of claim 13, wherein the crystalline form is hydrate.

19. The polymorph Form F of claim 18, wherein the hydrate is a monohydrate.

20. A composition or drug substance comprising (a) any one of the polymorph Form D, Form E or Form F of varenicline tartrate and (b) a crystalline, hydrate, solvate, amorphous, polymorph Form A, Form B, Form C, or other polymorphic forms of varenicline tartrate other than any one of the polymorph Form D, Form E or Form F, respectively, wherein the total weight of varenicline tartrate in the composition or drug substance is the sum of (a) and (b).

21. The composition of claim 20, wherein the composition comprising less than 0.1% to at least 99.9% by weight of any one of the polymorph Form D, Form E or Form F based on the total weight of varenicline tartrate in the composition.

22. An amorphous form of varenicline tartrate.

23. An amorphous form varenicline tartrate, characterized in that its X-ray powder diffraction pattern lacks discernible or sharp peaks and being substantially same as depicted in FIG. 26.

24. A pharmaceutical composition comprising any one of amorphous form, crystalline Form D, Form E or Form F of varenicline tartrate with one or more pharmaceutically acceptable carriers, excipients, diluents, additives, fillers, lubricants or binders.

25. The pharmaceutical composition of claim 24, wherein varenicline tartrate comprising less than 0.1% to at least 99.9% by weight of polymorph Form D based on the total amount of varenicline tartrate in the pharmaceutical composition.

26. The pharmaceutical composition of claim 24, wherein varenicline tartrate comprising less than 0.1% to at least 99.9% by weight of polymorph Form E based on the total amount of varenicline tartrate in the pharmaceutical composition.

27. The pharmaceutical composition of claim 24, wherein varenicline tartrate comprising less than 0.1% to at least 99.9% by weight of polymorph Form F based on the total amount of varenicline tartrate in the pharmaceutical composition.

28. The pharmaceutical composition of claim 24, wherein varenicline tartrate comprising less than 3% to at least 99.9% by weight of amorphous form based on the total amount of varenicline tartrate in the pharmaceutical composition.
